(19) United States
(12) Patent Application Publication
(10) Pub. No.: US 2026/0294536 A1
(43) Pub. Date: Oct. 1, 2026

(54) DEVICE AND METHOD FOR HIGHLY EFFICIENT HARD AND SOFT TISSUE ABLATION

(71) Applicant: IPG PHOTONICS CORPORATION, Marlborough, MA (US)

(72) Inventors: Gregory ALTSHULER, Lincoln, MA (US); ILYA YAROSLAVSKY, North Andover, MA (US); Viachelsav BRUNOV, Marlborough, MA (US); Anastasiya KOVALENKO, Wellesley, MA (US); Viktoriya ANDREEVA, Marlborough, MA (US)

(21) Appl. No.: 19/479,649

(22) PCT Filed: Apr. 26, 2024
(86) PCT No.: PCT/US2024/026490

§ 371 Date: Oct. 28, 2025

Related U.S. Application Data
(60) Provisional application No. 63/462,588, filed on Apr. 28, 2023.

(51) Int. Cl. A61B 18/26; A61B 17/00; A61B 18/00

(52) U.S. Cl. CPC A61B 18/26; A61B 2017/00119; A61B 2017/00199; A61B 2018/00678; A61B 2018/00702

(57) ABSTRACT

Systems and method for use in a laser lithotripsy procedure. The system can include a laser source configured to generate pulsed laser energy, an optical fiber configured to direct the pulsed laser energy at a target, and a controller. The controller is configured to receive input parameters, determine at least one procedure parameter, and calculate an N-factor (Nf) that corresponds to a number of laser pulses delivered to a treatment zone on the target and having a diameter approximately equal to a diameter of a laser crater created by the pulsed laser energy. Nf is compared to a maximum N-factor value (Nfmax) and in response to a comparison a laser operating parameter is either adjusted or is used to control the laser source.

### RELATED APPLICATIONS

The present application claims priority to U.S. Provisional Application Ser. No. 63/462,588, titled “DEVICE AND METHOD FOR HIGHLY EFFICIENT HARD AND SOFT TISSUE ABLATION,” filed on Apr. 28, 2023, the content of which is hereby incorporated by reference in its entirety.

### BACKGROUND

### Technical Field

The technical field relates generally to laser lithotripsy, and more specifically to a laser system configured to determine optimal laser pulse parameters for a laser lithotripsy procedure.

### Background Discussion

### INTRODUCTION

Laser lithotripsy is one of the most effective and less invasive urinary surgery procedures for stone disease treatment, including kidney, bladder, and ureter stones. Directed laser energy is guided from a laser source to target the stone (e.g. a uric stone, a calcium oxalate monohydrate stone, a cysteine stone, or the like) via optical fiber. Usually the surgeon uses a flexible or rigid/semi-rigid scope (endoscope, ureteroscope, cystoscope, renoscope, nephroscope, or the like) with a built-in-camera and illuminating source to guide the distal end of the fiber toward the target (stone, calculi, etc.) inside the patient's body.

In order to treat natural calculi, the laser energy power density should exceed the threshold of the calculi ablation (i.e., the ablation threshold). The ablation threshold is inversely proportional to the calculi coefficient of absorption. Therefore, Ho:YAG, Tm:YAG and thulium pulsed fiber lasers are the most efficient in laser lithotripsy because the wavelengths of these lasers (2.1, 2.01, 1.94 μm, respectively) match the peaks of the absorption spectrum of water which is the dominant chromophore in the near-IR wavelength range.

When a surgeon is assured that the distal end of the fiber is positioned in front of the target at an effective distance, e.g., when the distance between the target and fiber tip is less than 1 mm, he or she turns on or otherwise activates the laser in order to treat the stone.

Laser treatment is still a process of multiple complex factors. During stone treatment, several processes may occur simultaneously or one after another: 1) laser pulses overheat and vaporize the water in front of the distal end of the fiber, vaporized bubbles occur on the top of the fiber tip and start to grow, the bubble reaches the stone surface (so-called “Moses effect” or “Moses channel”), 2) laser pulses overheat and ablate the stone surface and create a laser crater, 3) small stone micro-particles formed as a part of the product of laser ablation elapse in all directions including the direction of the fiber tip, and the laser beam is absorbed and scattered by these particles to create so-called “debris screening” of the bottom of the laser crater from the laser beam, 4) the stone moves away from the fiber tip due to the water pressure wave and jet effect of ablated particles (so-called “retropulsion effect”), 5) stone craters and fragmentation occurs with stone cracks in larger particles and into finer particles or stone dust, the fiber tip overheats and becomes damaged, 6) if the laser pulse repetition rate falls below a threshold level, the bubble collapses and a gap between the fiber tip and the target fills back up with water.

The stone ablation process and its efficiency depends on altering and changing factors (parameters), such as (and not limited to): laser wavelength, pulse energy, peak power, pulse shape, pulse width, pulse frequency (repetition rate), average power, fiber diameter, the gap between the fiber tip and the target surface, target type (stone type/chemical composition and structure/shape of the stone), condition of fiber tip, surgical instrument type (rigid, semi-rigid, flexible endoscope) and speed of instrument manipulation (movement) or scanning speed inside the body (depends on surgeon and his or her skills and style), and many others.

Performing the operation in a body (as well as the location within the body) also has an effect on process efficiency: in order to not overheat and damage internals (e.g., tissue and organs), from a safety perspective the average laser power should be much less inside the ureter than, for example, inside the bladder or kidney because of the free space volume inside each organ. If the endoscope supports water flow, the flow rate is also a factor of influence.

Conventionally, a surgeon sets laser parameters (pulse energy, pulse width, pulse frequency etc.) manually, through trial and error, based on his or her clinical experience and the ability of the selected laser system, and based on literature data and advice of colleagues. Often the chosen parameters are sub-optimal and far from obtaining a high ablation efficiency. There is often no clear and obvious visible relationship between the laser settings and the effect on the stones being ablated.

### Literature Data

Numerous literature sources propose exact (predetermined) optimal laser parameters for different types of ablation modes (fragmentation, dusting, popcorning) and their dependence on laser type (Ho:YAG, Thulium Fiber Laser (TFL)), stone type (artificial Bego stone, COM, Uric acid etc.), organ type (environment), and fiber diameter. FIGS. 1A, 1B, and 1C exhibit a summary of various examples of these sources, and Table 1 below lists a bibliographic reference to each example.

TABLE 1
Bibliographic references for literature cited in FIGS. 1A, 1B, and 1C

| No. | Bibliographic reference |
|---|---|
| 1 | Klaver, P. et al., “In Vitro Comparison of Renal Stone Laser Treatment Using |
| | Fragmentation and Popcorn Technique”, Lasers in Surgery and Medicine, |
| | September 2017, Vol. 49(7): 698-704 |
| 2 | Elhilali, M. et al., “Use of the Moses Technology to Improve Holmium Laser |
| | Lithotripsy Outcomes: A Preclinical Study, Journal of Endourology, June 2017, Vol. |
| | 31(6): 598-604 |
| 3 | Aldoukhi, A. H. et al., “Watch Your Distance: The Role of Laser Fiber Working |
| | Distance on Fragmentation When Altering Pulse Width or Modulation”, Journal of |
| | Endourology, February 2019, Vol. 33(2): 120-126 |
| 4 | Winship, B. et al., “Dusting Efficiency of the Moses Holmium Laser: An Automated |
| | In Vitro Assessment”, Journal of Endourology, December 2018, Vol. 32(12): |
| | 1131-1135 |
| 5 | Vassar, G. J. et al., “Holmium: Yag Lithotripsy Efficiency Varies with Energy |
| | Density”, The Journal of Urology, August 1998, Vol. 160: 471-476 |
| 6 | Sea, J. et al., “Optimal Power Settings for Holmium: YAG Lithotripsy, The Journal |
| | of Urology, March 2012, Vol. 187: 914-919 |
| 7 | Mues, A. C. et al., “Quantification of Holmium: Yttrium Aluminum Garnet Optical |
| | Tip Degradation”, Journal of Endourology, September 2009, Vol. 23(9): 1425-1428 |
| 8 | Aldoukhi, A. H. et al., “Calyceal Fluid Temperature During High-Power Holmium |
| | Laser Lithotripsy in an In Vivo Porcine Model, Journal of Endourology, August |
| | 2018, Vol. 32(8): 724-729 |
| 9 | Black, K. M. et al., “Pulse modulation with Moses technology improves popcorn |
| | laser lithotripsy”, World Journal of Urology, June 2021, Vol. 39(6): 1699-1705 |
| 10 | Aldoukhi, A. H. et al., “Understanding the Popcorn Effect During Holmium Laser |
| | Lithotripsy for Dusting”, Urology, December 2018, Vol. 122: 52-57 |
| 11 | Panthier, F. et al., “Laser Fiber Displacement Velocity during Tm-Fiber and |
| | Ho: YAG Laser Lithotripsy: Introducing the Concept of Optimal Displacement |
| | Velocity”, J Clin Med, December 2021, 11(1): 181 |

For example, for a dusting ablation operating mode, the proposed laser parameters are in a range of the following: 10-28 W for average power, 0.05-0.5 J for pulse energy, and 10-80 Hz for pulse frequency. Nevertheless, few sources explain why these ranges of proposed laser parameters are actually optimal. Generally, the explanation is based on held ablation experiments without any nexus to the nature and/or reasons of stone ablation. Currently there is no clear methodology proposed for determining optimal laser parameters that have a dependence on different changing factors (circumstances) whether for real-time processes (during surgery) or prior to the procedure.

Applicant asserts that the published recommendation data contradicts itself and is difficult to use for the purpose of providing the best laser treatment.

There have been some efforts to increase the efficiency of laser lithotripsy in general and stone ablation efficiency in particular. Non-limiting examples of these efforts include: using real-time sensory feedback (e.g., sensor for determining the distance to target, tissue type sensor, stone type and size sensor and the like), improvement of fiber scanning by automatic periodical movement, applying specially shaped laser pulses or modulated periodic group of pulses, using presets for each specific stone and area/organ of operation etc. For example, some laser systems can support certain presets of parameters.

Some laser systems for lithotripsy can utilize so-called smart real-time feedback system in order to increase lithotripsy efficiency. For example, in Chinese patent CN110811826, titled Intelligent Lithotripsy System, an intelligent system implements a distance sensor (based on an ultrasonic distance sensor or a laser distance measuring device or a structured light distance measuring device), temperature sensor, and pressure sensor. Also, stone composition, structure, color, hardness, degree of stone fragmentation and the environment of the lesion is automatically determined through image recognition technology by negative feedback and automatically matched with a large database to provide the surgeon with the appropriate laser pulse energy, laser pulse time, and other parameters for the surgeon's reference. This configuration utilizes a large data platform and artificial intelligence deep learning system that proposes efficient laser parameters for laser lithotripsy.

In PCT publication WO2013154708, titled Surgical Laser Systems and Laser Lithotripsy Techniques, a stone analyzer comprises an imager, and Laser Doppler Vibrometer (LDV) or Laser Induced Breakdown Spectrometer (LIBS) to determine characteristics of a targeted stone (such as the stone's size and geometry, composition of the targeted stone). Received feedback is mapped to laser energy settings (e.g., a pulse repetition rate, pulse width, etc.) for generating laser energy tuned to fragment the targeted stone. In some embodiments, the laser controller determines the laser treatment to be performed to fragment the stone based on the identified type of stone.

In PCT publication WO2021026161, titled Laser Control Using a Spectrometer, a surgical feedback control system based on a spectrometer comprises a feedback analyzer configured to receive a reflected signal from a target in response to electromagnetic radiation directed at a target, and a controller that can perform a predetermined operation based upon the received reflected signal, including determining a composition of the target, or programming a laser setting to direct laser energy to the target. The system continuously identifies the composition of a target through an endoscope and updates the laser settings throughout a procedure. The spectroscopic system collects information about the target materials that is useful for diagnostic purposes, and for confirming that laser parameters are optimal for the target. A feedback analyzer may automatically optimize an operation mode of the laser system (using an optimal setup database library) and reduces the risk of human error.

The controller of the laser system in PCT publication WO2021026164, titled Endoscopic Laser Energy Delivery System and Methods of Use, may automatically program laser therapy with appropriate laser parameter settings due to target composition based on a machine learning algorithm trained with spectroscopic data.

In PCT publication WO2016201092, titled Bodily Substance Detection by Evaluating Photoluminescent Response to Excitation Radiation, intelligent feedback is used for detecting human stones and the distance to the stone by evaluating the photoluminescence radiation level response (fluorescence radiation) emitted by stones excited by a low-power probe laser in order not to perforate walls of the urinary system. The method also includes one or more parameters of the delivery of the ablation energy that are adjusted based on received parameters of the detected photoluminescence radiation.

In PCT publication WO2019157406, titled System, Method and Computer-Readable Storage Device for Controlling Laser Light Source of Lithotripsy Device, a laser controller system sorts out and changes one variable operating parameter of the laser source of the lithotripsy device (energy, peak power, pulse width, the average power, and the frequency of the laser light output from the laser source) during a stone ablation process and then determines which of the plurality of basic settings is more suitable for breaking or rupturing another layer of the stone. User assistance is still needed on reaching a conclusion of increasing of ablation efficiency in each particular case.

In PCT publication WO2015175151, titled Computer Aided Image-Based Enhanced Intracorporeal Lithotripsy, a processor of a laser system is programmed to execute the image processing routine and use the analysis program to determine the characteristics of the stone. The system utilizes database time-varying characteristics of the current stone that is accumulated from the start of the ablation. Using these characteristics, a processor calculates the optimal power parameters and sends it to a controller, which adjusts the power settings in response to one or more energy generation parameters.

In US Patent U.S. Pat. No. 11,160,573, titled Scanning Ureteroscope for Maximizing Efficiency in Laser Lithotripsy, periodic movement of the rocker arm of the scope (scanning) is coordinated with the laser pulse by a computer such that laser energy from each pulse is irradiated to two different areas of the stone rather than to one area so that a hole is continuously bored. This approach leads to more laser energy being spent on breaking the stone rather than heating the water.

There are laser systems that use a pre-determined amplitude periodic group of pulses for “shaped pulse laser lithotripsy” to provide a high ablation rate while also minimizing retropulsion of the ablation products, such as described in Applicant's PCT publication WO2020033121, titled Method and Apparatus for Laser Lithotripsy. Proposed presets are divided into different stages of stone fracturing depending on the treatment target stone size: fragmentation, dusting, and popcorning. For achieving a minimum retropulsion effect combined with a high ablation efficiency, it was proposed to use an extended pulsewidth or use a dual pulse regime (modulation or periodic variation of the pulse energy, peak power, and pulse frequency, and formation of optimal pulse shapes). Pulse energy is separated such that water is evaporated to form a bubble (Moses channel) and also pulse energy is used to ablate the stone. High power in the first pulse is not needed, which would overheat the water and create a force wave pressure that leads to retropulsion of ablation products. Once the Moses channel is overcome, the pulse power can be increased to increase ablation.

There are approaches to analyze the large arrays of data received during laser lithotripsy and to implement a formula of analytical approximation based on received data to calculate different parameters of procedure. For example, in order to increase lithotripsy efficiency for stones located in the upper third of the ureter, one can calculate a coefficient of risk of “proximal migration”(retropulsion of the stone or its fragments due to dilation of the ureter proximal to the stone, the pressure of the irrigation fluid, and the effect of a laser on the ureteral mucosa) for selecting optimal therapeutic approach by using a proposed empirical formula, such as the one disclosed in Russian patent RU2725961, titled Method of Choosing Therapeutic Approach in Laser Contact Ureterolithotripsy of Stones of Upper Third of Ureter, and expressed as:

$$Y = exp ( b 0 + b 1 * X 1 + b 2 * X 2 - b 3 * X 3 + b 4 * X 4 + b 5 * X 5 ) / \text{} [ 1 + exp ( b 0 + b 1 * X 1 + b 2 * X 2 - b 3 * X 3 + b 4 * X 4 + b 5 * X 5 ) ] ,$$

where
Y=coefficient of risk of proximal migration,
b0-b5=determined regression coefficients,
X1=the distance from the stone,
X2=the maximum size of the stone,
X3=the minimum size of the stone,
X4=presence of hydronephrosis as hydrostatic dilation of the renal pelvis and calyces as a result of obstruction to urine flow downstream (if absent-0, if present-1),
X5=the duration of the stone's standing (if up to 30 days-0, more than 30 days-1). If the value of the risk coefficient Y is 30% or less, it is suggested to perform lithotripsy using a rigid ureteroscope, if the value of the risk factor Y is more than 30%, it is suggested to perform lithotripsy with a flexible ureteroscope.

The proper choice of laser lithotripsy parameters allows for the ability to provide safe and fast stone removal which simultaneously requires:
1. Prevent thermal damage to the kidney, ureter or bladder due to overheating of water which is dependent on the average laser power and water irrigation and outflow.
2. Prevent perforation of the wall of kidney, ureter or bladder due to soft tissue ablation. The chance of perforation increases with laser pulse energy and repetition rate.
3. Minimize the time of laser treatment which depends on ablation efficiency and the amount of time the laser is on during treatment. This is especially important for large stones due to limited time of anesthesia.

### SUMMARY

Aspects and embodiments are directed to a method and system for use in a laser lithotripsy procedure.

In accordance with an exemplary embodiment, a system for use in a laser lithotripsy procedure is provided that includes a laser source configured to generate pulsed laser energy, an optical fiber configured to direct the pulsed laser energy at a target, and a controller coupled to the laser source and configured to: receive one or more input parameters, determine at least one procedure parameter, wherein the at least one procedure parameter includes one or more laser operating parameters, calculate an N-factor (Nf) based at least in part on one or more procedure parameters and at least one input parameter, where Nf corresponds to a number of laser pulses delivered to a treatment zone having a diameter approximately equal to a diameter of a laser crater (Dc) created by the pulsed laser energy that is directed at the treatment zone on the target, compare Nf to a maximum Nf (Nfmax), and in response to a determination that Nf is greater than Nfmax, adjust at least one laser operating parameter of the one or more laser operating parameters, or in response to a determination that Nf is less than or equal to Nfmax, control the laser source using the one or more laser operating parameters.

In accordance with another exemplary embodiment, a method for performing a laser lithotripsy procedure is provided that includes providing a controller configured to: receive one or more input parameters, determine at least one procedure parameter, wherein the at least one procedure parameter includes one or more laser operating parameters, calculate an N-factor (Nf) based at least in part on one or more procedure parameters and at least one input parameter, where Nf corresponds to a number of laser pulses delivered to a treatment zone having a diameter approximately equal to a diameter of a laser crater (Dc) created by the pulsed laser energy that is directed at the treatment zone on the target, compare Nf to a maximum Nf (Nfmax), and in response to a determination that Nf is greater than Nfmax, adjust at least one laser operating parameter, or in response to a determination that Nf is less than or equal to Nfmax, control a laser source using the one or more laser operating parameters.

In one example, the one or more laser operating parameters comprise pulse energy, pulse frequency, average power, peak power, and/or pulse width.

In one example, when the controller determines that Nf is greater than Nfmax, the controller is configured to adjust the at least one laser operating parameter such that Nf is less than or equal to Nfmax.

In one example, the controller is further configured to: display Nf on a display device coupled to the controller, and in response to the determination that Nf is greater than Nfmax, output a negative alert message on the display device, or in response to the determination that Nf is less than or equal to Nfmax, output a positive alert message on the display device.

In one example, the one or more procedure parameters used to calculate Nf include laser pulse frequency, laser crater diameter (Dc), and an optical fiber speed (ν). In a further example, Nf is calculated according to the formula: Nf=(f*D_{c})/ν, where f(Hz) is the laser pulse frequency, D_{c} (mm) is the laser crater diameter, and ν is the speed of optical fiber that directs the pulsed laser energy at the treatment zone.

In one example, the laser crater diameter D_{c} is in a range according to the following expression: (2NAΔ+d) (0.12⁴\sqrt{Pₚₑₐₖ} In (E₁/(0.25 π F′(2NAΔ+d)²))—\sqrt{Pₚₑₐₖ}/250)+(1.82d) (0.21−0.025 E₁)≤D_{c}≤(2NAΔ+d) (1/(1.61+10.23d⁴+(0.92−2.97d)Δ))×(1+(\sqrt{Pₚₑₐₖ}/12.85) ln ((12.85//\sqrt{Pₚₑₐₖ}) (1/(6.4−6.75d)) (E/(0.25 π F′(2NAΔ+d)2−1)+1)), where NA is a fiber numerical aperture, A (mm) is a gap between a distal tip of the fiber and the stone surface, d (mm) is a core diameter of the fiber, Pₚₑₐₖ (W) is a peak power of the laser pulse, E₁ (J) is a single pulse energy, and F′(J/cm²) is a threshold of stone ablation.

In one example, the controller is configured to calculate Nfmax, where Nfmax corresponds to a number of laser pulses delivered to the treatment zone having a diameter approximately equal to the laser crater diameter D_{c} that provides an average calculi ablation efficiency per pulse for a maximum number of pulses that is greater than or equal to a predetermined K value corresponding to an ablation efficiency achieved after impact from a single (first) pulse.

In one example, the K value is in a range of about 25-75% inclusive. In a further example, the K value is in a range of about 25-50% inclusive.

In one example, the controller is configured to calculate Nfmax, wherein Nfmax is calculated according to the formula: Nfmax=(12.4−3.4d)+(3.3d−8.4)/(1+4.7×10⁻⁵ exp (Pₚₑₐₖ/0.0375))−((14.7d−16)+(80−65d) Pₚₑₐₖ+(55d−62)Pₚₑₐₖ²)/(1+0.34 E^{5.5-2.6 Ppeak}). where Pₚₑₐₖ is peak power.

In one example, Nfmax is in a range of 1-12 inclusive. In a further example, Nfmax is in a range of 1-7 inclusive. In a further example, Nfmax is in a range of 1-5 inclusive. In a further example, Nfmax is in a range of 1-3 inclusive.

In one example, the controller is configured to display on a display device at least one laser operating parameter based on the comparison.

In one example, the controller is configured to display on a display device at least one of Nf and Nfmax.

In one example, the system further includes at least one sensor coupled to the controller and configured to measure at least one input parameter and/or at least one procedure parameter. In a further example, the at least one sensor includes a sensor configured to measure a fiber speed.

In one example, when the controller determines that Nf is greater than Nfmax, the controller is configured to adjust the at least one laser operating parameter such that Nf is less than or equal to Nfmax.

In one example, the controller is further configured to: in response to a determination that Nf is greater than Nfmax, output a negative alert message that comprises at least one of an audio alert message, a visual alert message, and a haptic alert message, or in response to a determination that Nf is less than or equal to Nfmax, output a positive alert message that comprises at least one of an audio alert message, a visual alert message, and a haptic alert message.

In one example, the controller is configured to output an alert message that comprises at least one of an audio alert message, a visual alert message, and a haptic alert message in response to the comparison. In a further example, the system further includes a user input device that is coupled to the controller, wherein the user input device is configured to receive input from a user, the input including at least one value for a laser operating parameter that is used by the controller to control the laser source. In a further example, the alert message includes information to the user regarding whether Nf is greater than Nfmax or whether Nf is less than or equal to Nfmax.

In one example, the input parameters comprise at least one of target parameters, system parameters, and safety parameters. In one example, the input parameters include target parameters and the target parameters comprise target type, target location, and one or more target characteristics. In one example, the target is calculi and the one or more target characteristics comprise size and/or hardness of the calculi. In one example, the input parameters include at least one system parameter and the at least one system parameter comprises a fiber diameter and/or a fiber numerical aperture. In one example, the input parameters include at least one safety parameter and the at least one safety parameter comprises maximum average power, maximum peak power, maximum pulse energy, and/or maximum pulse frequency.

In one example, the method further includes measuring at least one input parameter and/or at least one procedure parameter using at least one sensor. In a further example, the at least one input parameter includes fiber speed.

In one example, the controller is further configured to receive input from a user input device, the input including at least one value for a laser operating parameter that is used by the controller to control a laser source.

In accordance with another exemplary embodiment, a system for use in a laser lithotripsy procedure is provided that includes a laser source configured to generate pulsed laser energy, an optical fiber configured to direct the pulsed laser energy at a target, and a controller coupled to the laser source and configured to: receive one or more input parameters, determine at least one procedure parameter, wherein the at least one procedure parameter includes one or more laser operating parameters, calculate an N-factor (Nf) based at least in part on one or more procedure parameters and at least one input parameter, where Nf corresponds to a number of laser pulses delivered to a treatment zone having a diameter approximately equal to a diameter of a laser crater (D_{c}) created by the pulsed laser energy that is directed at the treatment zone on the target, display Nf on a display device coupled to the controller, compare a laser operating parameter to a threshold value, and in response to a determination that the laser operating parameter is greater than the threshold value, adjust at least one laser operating parameter of the one or more laser operating parameters, or in response to a determination that the laser operating parameter is less than the threshold value, control the laser source using the one or more laser operating parameters.

In one example, the laser operating parameter that is compared to a threshold value is a pulse energy. In one example, the controller is further configured to calculate the pulse energy based at least in part on a pulse frequency. In one example, the controller is further configured to calculate the pulse frequency based at least in part on a maximum N-factor (Nmax).

In one example, the controller is configured to adjust the at least one laser operating parameter such that Nf is determined to be less than or equal to a maximum N-factor Nfmax.

In one example, in response to a determination that the laser operating parameter is greater than the threshold value, the controller is configured to adjust a laser operating parameter value to a maximum safety value associated with the laser operating parameter.

In one example, the controller is configured to display at least one laser operating parameter based on the comparison.

Still other aspects, embodiments, and advantages of these example aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Embodiments disclosed herein may be combined with other embodiments, and references to “an embodiment,” “an example,” “some embodiments,” “some examples,” “an alternate embodiment,” “various embodiments,” “one embodiment,” “at least one embodiment,” “this and other embodiments,” “certain embodiments,” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIGS. 1A, 1B, and 1C are tables of laser settings for lithotripsy procedures according to various literature sources;

FIG. 2 is a table showing typical speeds of fiber movement for different scope types and human organ types;

FIG. 3 is a schematic representation of the N-factor as a number of pulses in one spot/point in accordance with various aspects of the invention;

FIG. 4 is a table showing safe maximum value for various laser operating parameters for different body organs in accordance with aspects of the invention;

FIG. 5 are photographs of crater arrays on the surface of Bego stone as a function of pulse energy and number of pulses for different peak powers in accordance with aspects of the invention;

FIG. 6 is a photograph and diagram of a surface of a Bego stone and its measured cross-section profile in accordance with aspects of the invention;

FIG. 7 shows several graphs of depth profiles of craters on the surface of Bego stone for various pulse energies (single pulse, 500 W peak power) in accordance with aspects of the invention;

FIG. 8 shows a table of measured crater diameters after impact from a single pulse for different fiber sizes for various gaps (distance between fiber and stone), pulse energies, and peak powers in accordance with aspects of the invention;

FIG. 9 shows a table of measured saturated crater diameters after impact from multiple pulses for different fiber sizes as a function of gap, pulse energy, and peak power in accordance with aspects of the invention;

FIG. 10 are photographs of groove arrays on the surface of Bego stone as a function of average power for different fiber scanning speeds in accordance with aspects of the invention;

FIG. 11 is a table of measured specific crater volumes in a scanning mode as a function of peak power, pulse energy, and N (pulse sequence number) in accordance with aspects of the invention;

FIG. 12 is a pair of tables showing a comparison between measured and calculated crater diameters after impact from single and multiple pulses in accordance with aspects of the invention;

FIG. 13 is three separate graphs showing a comparison between measured and calculated crater diameters as a function of pulse energy for different gaps between Bego stone and the fiber tip in accordance with aspects of the invention;

FIG. 14 is three separate graphs showing a comparison between measured and calculated saturated crater diameters as a function of pulse energy for different gaps between Bego stone and the fiber tip in accordance with aspects of the invention;

FIG. 15 shows three graphs of a calculated crater volume for 0.2 mm fiber for different (pulse sequence number) as a function of peak power and pulse energy in accordance with aspects of the invention;

FIG. 16 is a table showing measured vs calculated values for the most efficient number of pulses in one spot for different fiber diameters as a function of peak power and pulse energy in accordance with aspects of the invention;

FIG. 17 is three graphs showing the most efficient N-factor as a function of pulse energy and peak power for different fiber diameters in accordance with aspects of the invention;

FIG. 18 is a flowchart showing steps in one example of a smart assistant mode of operation (“first” approach) in accordance with aspects of the invention;

FIG. 19 is a flowchart showing steps in another example of a smart assistant mode of operation (“second” approach) in accordance with aspects of the invention;

FIG. 20 is a flowchart showing steps in another example of a smart assistant mode of operation (“third” approach) in accordance with aspects of the invention;

FIG. 21 includes two screen shots from a GUI showing examples of notifications that can be displayed on a laser system display device screen to a user in regard to the N-factor in accordance with aspects of the invention;

FIG. 22 is a block diagram of a lithotripsy system in accordance with aspects of the invention; and

FIG. 23 is a block diagram of a configuration for pulsed laser energy output in accordance with aspects of the invention.

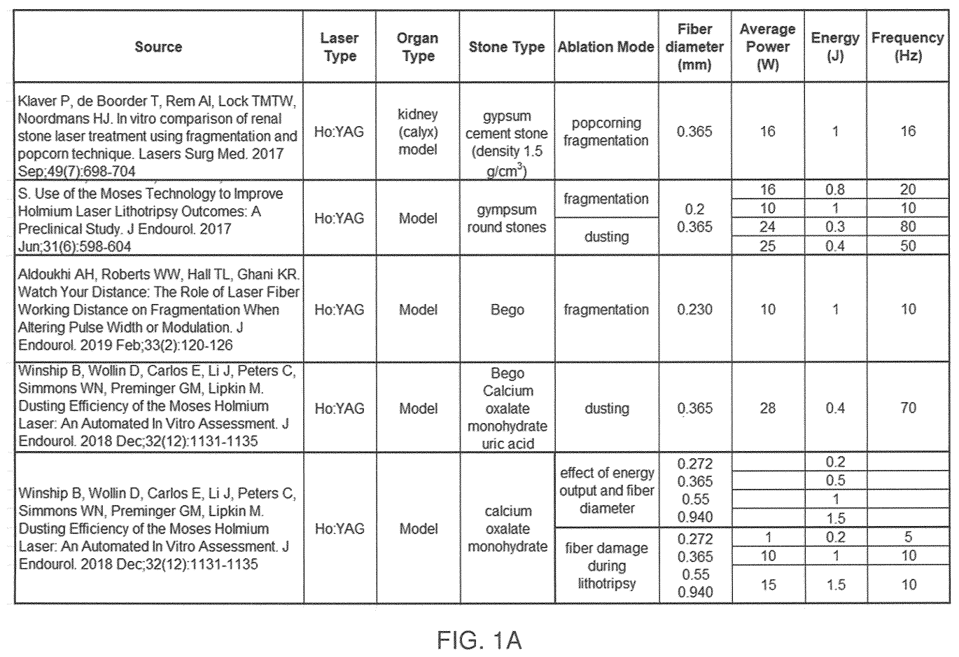

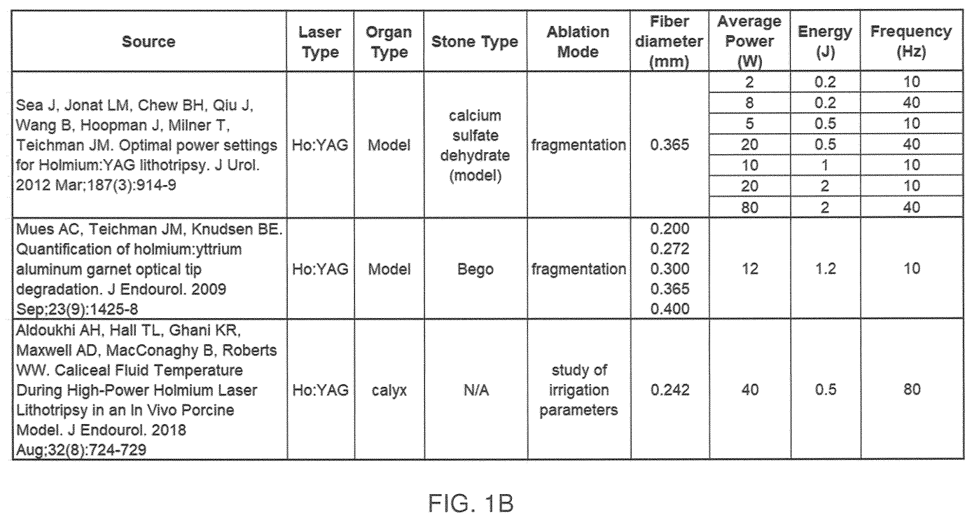

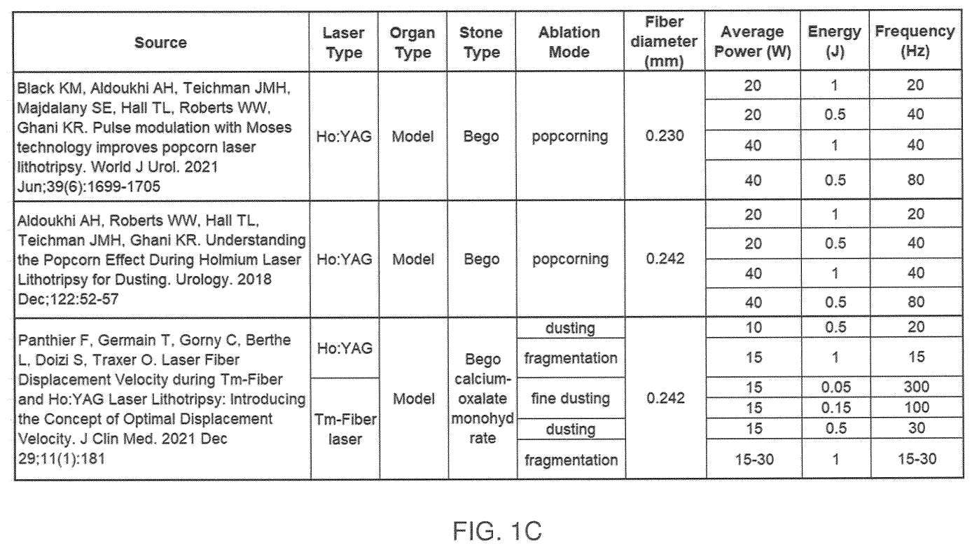

### DETAILED DESCRIPTION

### Physics and Nature of the Ablation Process

After the formation of the Moses channel between the fiber tip and the targeted stone, laser energy begins to impact mostly the stone and create the beginning of the ablation process. The irradiated area on the surface of the stone depends on the fiber output aperture and the distance to the target surface. The farther away the stone is and the greater the size of the fiber output aperture, the larger the irradiated area and the lower the laser power density on the surface of the stone. However, if the ablation process were to be only tied with the dissociation of material in the irradiated area when the laser power density exceeds the destruction threshold of the stone, then the produced crater or cavity on the surface of the stone should correspond (match, fit) an irradiated area that can be calculated according to:

$$D = 2 * NA * L + d \qquad ( 1 )$$

where
D is the diameter of the irradiated area on the surface of the stone,
NA is the output numerical aperture of the fiber,
L is the distance to the stone surface, and
d is the fiber core diameter (size of emitting source).

If the distribution of laser power density at the fiber tip is uniform and the laser power density remains uniform on the stone surface, exceeding the destruction threshold of the stone, then the produced ablation crater on the stone is reasonably assumed to be the same as the irradiated area. If this distribution on the stone surface is non-uniform (e.g. Gaussian), then it follows that the produced ablation crater should be the same as the irradiated area where the laser power density is equal to or greater than the destruction threshold of the stone. It follows that the ablation crater can be the same or less than the irradiated area. According to some conventional reasoning, there is no obvious reason that the ablation crater could be greater than the irradiated area in either case. Moreover, it is reasonable to assume that there will be no correlation of the ablation crater area with the number of irradiated pulses. An increase in number of pulses onto one stone point may be associated with an increase in the depth of the crater and an increase in distance between the fiber tip and the stone surface. Retropulsion impact may also increase fiber tip to stone distance. In both cases the result is decreased laser power density on the stone surface.

However, as practice and experiments show, the ablation crater diameter is usually larger than the irradiated area, even for a single laser pulse. For example, when using a pulsed thulium laser with a 1940 nm wavelength and 500 W peak power, a 200 μm core fiber situated in contact with the stone and surrounded by water (the distance to the target is shorter than 100 μm) creates an ablation crater having a diameter of about 1 mm after impact with a single 6 J pulse on the surface of stone, which is five times more than the fiber diameter.

This shows that the nature of the ablation process is more complicated than just the dissociation of material in a laser-irradiated area.

The physics/nature of the ablation process is dependent on many factors: laser parameters such as irradiation wavelength, average and peak power, pulse width, energy and frequency, shape of laser pulses, etc., also referred to herein as laser operating parameters, utilizing instrument (scope type, fiber diameter, fiber tip condition, speed of fiber movement etc.), nature of the stone (chemical composition, structure, size, absorption, etc.), environment features (distance to the target, the substance in the interlayer between fiber tip and the stone surface, organ type, amount of the stones etc.), and others. These varying parameters affect various physical processes and mechanisms of ablation including one or more of the following: photomechanical mechanisms, photothermal mechanisms, and photochemical or photothermo-chemical mechanisms as described in Applicant's PCT publication WO2020033121.

The photothermal mechanism is the absorption of radiation energy by the target material that is converted to thermal energy that then produces thermo-mechanically induced stresses in the stone within and possibly, by conduction, around the irradiated area: the local increase in temperature causes thermal expansion of the stone material or vaporization of entrapped water in the stone that results in high pressures and mechanical stresses that can exceed the tensile stone strengths. Note that there may be varying degrees of tensile strengths (crystal binding or cross-linking for large versus small domains) throughout any given stone. The thermo-mechanical mechanism also comes into play when cavitation bubbles collapse near the stone surface resulting in formation of microscopic cracks and other defects in the stone. The induced stress propagates as stress waves in and outside the irradiated zone. Such cavitating bubbles can be induced by a shock wave that is, in turn, produced by violent collapse of a large laser-induced bubble created via the photothermal process. A photothermo-chemical mechanism relates to the dissociation of molecular bonds inside the stone bulk based on absorption of high energy photons or thermally induced change in chemical composition which can induce changing of stone absorption of laser radiation and changing of mechanical property or properties such as mechanical strength. Thermal effects, for example, can lead to chemical reactions in or on the mineral matrix of the stone, such as pyrolysis, if there is organic material involved. The former process leads to direct mechanical damage to the stone whereas the latter produces cavitation bubbles that can mechanically damage a nearby stone.

These ablation mechanisms, together with an understanding of the structural and chemical composition of the stone, suggest that the ablation crater can be greater than the irradiated area, namely via stress wave propagation, thermal diffusion and/or stress propagation depending on the structural and chemical composition of the stone.

There are three surgical techniques that have been developed through experimentation involving the described mechanisms: A fragmentation mode technique utilizes lower frequency of pulses at higher peak laser power applied to one small area of the stone for a relatively long period of time. This technique results in relatively deep and large-area holes with macro-cracking produced by the photo-thermal and ensuing thermo-mechanical stresses. A dusting (scanning, dancing) mode technique utilizes a fiber continuously moving across the stone surface during delivery of higher frequency laser pulses. This technique ablates a thin layer of stone along the path traversed by the laser. Finally, a so-called popcorning mode technique utilizes a stationary fiber directed into a swarm of stone and fragments created by repetitive laser firing. Each pulse of the laser induces bubble expansion and collapse in surrounding water that, in turn, produces water flow in confined volume such as may be seen in a kidney's calyx or bladder. Each specific surgical technique may be efficient with one stone type and but less efficient with another. In other words, there is no universal all-purpose laser mode for all stone types and environments.

For a given type of fiber, one may deduce that the greater the laser impact (power density) applied to the stone, the greater the size of the ablation crater. However, this is not completely true as it neglects other consequences of an ablation process. According to one publication, [Boris Majaron et al., “Debris screening and heat diffusion in Er: YAG drilling of hard dental tissues” Proc. SPIE 2973, Lasers in Dentistry III, (15 May 1997)) the dependence of the ablation crater's depth and volume on the applied laser fluence, during human dentin and enamel exposure to Er: YAG laser pulses, exhibits a non-linear, quasi-logarithmical dependence. The process of laser drilling of stones in Lithotripsy can be similar to that in human dentin and therefore should also exhibit such nonlinearities. Debris screening nonlinearities are complex with multiple origins: Debris ejection and ablation effects on the stone can interfere with the current laser pulse if it is long enough, but may not interfere with the next laser pulse if the pulse frequency is low enough. Debris adherence to the fiber tip, on the other hand can be permanent and persist during the entire procedure unless the fiber is cleaved.

When the incident laser fluence of a single pulse at a given spot exceeds the threshold for stone destruction, the depth and volume of the ablation crater initially increases with increase in laser fluence (energy density). However, further increase in the laser fluence beyond a certain fluence level does not lead to further crater growth and therefore the additional laser energy is wasted. This fluence level can be determined and occurs because of so-called “debris screening” which is the screening (covering) of the fiber tip by the ejected debris-small micro-particles of the stone. This (very hot) debris is ejected in all possible directions and that portion directed to the tip can adhere to it or, at least, can be in the path of the laser during firing and partially obstruct energy delivery to the stone surface by scattering or absorbing radiation. The process of debris formation is non-linear, and is disproportionately more severe when greater laser energy is applied. One may therefore expect a disproportionate decrease in ablation rate with increase in laser power density (or fluence).

Moreover, if debris adheres to the fiber tip, it will continue to absorb laser energy and will transfer heat to the fiber. The fiber tip can become thermally damaged and can then also directly absorb the laser energy leading to additional fiber tip damage. At this point, most of the applied laser energy is blocked from impacting the stone and the clinician must remove the fiber from the endoscope, out of the human body, in order to cleave and initiate the fiber tip.

In summary, notwithstanding the detrimental effects of retropulsion, higher pulse energies can be ineffective and even adverse. On the other hand, pulse energies that are too low can decrease the size of an ablation crater prolonging the procedure or, in the worst case, can fall below the ablation threshold. Therefore, the most efficient pulse energy for a given peak power is somewhere between these two limits. Thus, it is important to better understand and to quantify the ablation process so that an optimum range of laser parameters may be found in order to optimize the ablation efficiency and, therefore, increase the safety and effectiveness of the lithotripsy surgical procedure.

### Ablation Rate and Ablation Efficiency

It has been shown that the efficiency of the stone ablation process can differ under changing factors related to laser parameters, environmental features, and target types. One can characterize the potential potency of an ablation process by the ablation rate and the ablation efficiency.

The ablation rate (A_{R}, mm³/s) is the speed of a target's volume removal or disassociation. The ablation rate quantifies how quickly the target can be ablated and removed. In other words, A_{R} quantifies, on average, how much volume (mm³) is deleted/removed per unit exposure time (e.g., one second). The ablation rate can be expressed as:

$${A}_{R} = V / t \qquad ( 2 )$$

where V (mm³) is the target volume, and t(s) is the time of full target laser irradiation for destruction/dissociation/removal. Put another way, the ablation rate shows how fast the current target with a certain volume can be destroyed, which impacts lasing time and operation (procedure) time. As described above, the ablation rate is expected to be a function of the laser parameter settings and the goal is to minimize this metric for a given procedure.

The ablation threshold, the ablation process and the competing deleterious effects of screening, retropulsion and tip damage all depend upon power density, which is proportional to the ratio of energy to time for a given fiber type and environment. This implies that there is a need to introduce an additional metric to properly characterize an ablation process in lithotripsy. While the ablation rate is an important metric to determine the time of a procedure for a given stone size, it is even more important to minimize the total laser energy required for a given procedure and stone. For example, there are situations where applying extra pulse energy does not lead to an increase in the ablation rate. For instance, at the same power density, 3 J could have the same ablation rate as 6 J. At a lower power density, the 3 J energy pulse would provide a higher ablation rate than the 6 J case. This can happen because of the competing effects discussed above and, possibly, other effects. There is no sensible reason to apply extra energy if the same ablation rate can be achieved with a lower pulse energy (energy efficiency).

The ablation efficiency (A_{E}, mm³/J) is the amount of stone volume (mm³) that is deleted/removed by applying laser energy to the target. It reflects the efficiency of the applied laser pulse energy. For example, the ablation efficiency of a single laser pulse is the volume of the resulting crater/cavity divided by the pulse energy. For multiple pulses applied to one target point, the ablation efficiency is the final crater volume divided by the total applied energy which is the sum of the energies of each applied pulse. The average ablation efficiency can be expressed as:

$${A}_{E} = V / \sum ( E ) \qquad ( 3 )$$

where Σ(E) (J) is the total applied laser energy: Σ(E)=E₁+E₂+ . . . +Eₙ (n=pulse sequence number or number of pulses). If the energy of each pulse is the same Eⱼ=E, for all j, then Σ(Eⱼ)=N*E where N is the number of pulses.

In other words, the ablation efficiency reveals the target volume that can be removed by applying a certain laser energy unit (for example, one Joule).

When applied to the lithotripsy procedure where the fiber may be moved relative to the stone, the ablation efficiency can be expressed as:

$${A}_{E} = ( S * υ ) / \overline{P} \qquad ( 4 )$$

where S (mm²) is the vertical cross-sectional area of the laser-produced crater after scanning, ν (mm/s) is the relative speed of fiber to the stone and P̅ (W) is the average laser power. Note that in certain circumstances the inverse of the ablation efficiency (1/A_{E}, J/mm³) may also be referred to as the ablation efficiency. In this case, the inverse indicates how much energy should be spent to ablate a unit target volume (for example, one cubic millimeter).

The ablation efficiency is important to evaluate during a procedure. If the ablation efficiency is known, one can evaluate the procedure's lasing time, T, with the following expression if the stone volume, V, is known:

$$T = V / ( {A}_{E} * \overline{P} ) \qquad ( 5 )$$

Taking into account expression (3), the lasing time may also be expressed as:

$$T = \sum ( E ) / \overline{P} \qquad ( 6 )$$

Expression (5) shows that increasing ablation efficiency shortens the lasing time and therefore procedure time which is critical for treating a large stone and finishing the procedure within the anesthesia time. In order to achieve this it is necessary to understand first what the ablation efficiency depends on, for example, what the substance of the target is, and what can be done to increase the ablation efficiency.

Ablation efficiency depends on many factors including laser wavelength, pulse energy, pulse frequency, pulse width, pulse shape, average and peak power, laser power density, fiber diameter, energy distribution inside the fiber, numerical aperture of the fiber, condition and shape of fiber tip, distance to the target, target type (stone type/chemical composition and structure/shape of the stone), target absorption, surgical instrument type (rigid, semi-rigid, flexible endoscope) and speed of instrument manipulation (movement), clinician techniques and other factors.

To illustrate, the ablation efficiency for a fiber situated in contact with the stone will be higher than for a fiber a distance of 1-2 mm away from the stone, all other factors equal, because less laser is absorbed in the medium (water) in the gap between the fiber tip and the stone and possibly because a higher power density on the surface of ablated stone (smaller laser spot size due to laser beam divergence can increase the ablation rate). In addition, fibers with greater diameters and apertures situated at the same distance to the stone, all other factors the same, deliver lower power density to the stone's surface and therefore may have a lower ablation efficiency, depending on the level of the power density relative to the ablation threshold. It is therefore important to competently select the proper power, energy and fiber type and to control the distance to the target in order to perform lithotripsy more efficiently.

As was discussed above in reference to drilling of human dentin, it is anticipated that a similar ablation efficiency nonlinearity may apply for multiple pulses delivered to the same point on a stone. The ablation efficiency of multiple, consecutive pulses to a single point or spot of a target stone is less than that for the first pulse to the point or spot. The crater created by the first (or previous) pulse increases the distance of the fiber to the stone surface (bottom of the crater) and therefore decreases the incident power density and fluence for the following (subsequent) pulse. Laser radiation of subsequent pulses will be attenuated due to many factors describe above including, but not limited to, longer distance between distal end of fiber tip and laser target providing opportunity for greater attenuation due to scattering and absorption by ablation products, attenuation due to ablation through deeper water-filled crater and other factors. Therefore, the number of pulses hitting one target spot of a stone and the effect on the ablation efficiency depends both on pulse frequency and on fiber speed of movement. The greater the fiber speed, the fewer the number of pulses that hit a given target point. In conventional procedures, urologists select only three laser parameter for treatment namely, laser energy, repetition rate and pulsewidth (or peak power). Their choice controls/determines the average laser power that is important to prevent surrounding soft tissue overheating and damage. The conventional understanding is that energy should be selected in the range to provide acceptable level of ablation efficiency per pulse, retropulsion level and visibility. Frequency (repetition rate) f is chosen to be as high as possible, maximal fmax, while keeping the average power, P=E*f, at a safe level Pmax to prevent thermal damage of soft tissue from overheated water. However this approach to increase rep rate is not effective for maximizing ablation efficiency and/or minimizing treatment time due to the above mentioned mechanisms that degrade ablation efficiency during multiple pulses to the same point on the target. For this reason Applicant proposes a new parameter, N-factor, to be considered for laser lithotripsy.

### N-factor

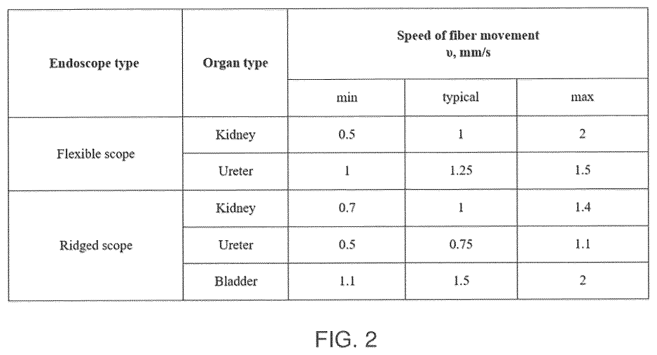

During a laser lithotripsy procedure, the clinician directs the laser pulses to the stone and moves the scope tube with the fiber positioned in front of the stone (dusting mode). The speed at which the fiber is moved depends on the clinician, the type of scope, and the body organ of treatment. For example, FIG. 2 displays a table showing typical fiber movement speeds for different scope and organ types.

The speed of the fiber and pulse frequency are parameters that determine the number of pulses to a spot on the target that, in turn, determine D_{c}, the crater diameter. Therefore fiber speed and pulse frequency determines the ablation efficiency. For purposes of this disclosure, the number of pulses that hit one target point or spot, called an “N-factor”(=Nf). N_{f} can be expressed as:

$$Nf = ( f * {D}_{c} ) / υ \qquad ( 7 )$$

where
f(Hz) is the laser pulse frequency (repetition rate),
D_{c} (mm) is the laser crater diameter, and
ν (mm/s) is the speed of the fiber relative to the point on the stone (i.e., the relative speed of movement of a distal tip of the fiber in a direction parallel to the stone surface).

Increasing the pulse frequency directly leads to an increase in the N-factor. Increasing the pulse energy at the current peak power (applied to one target point) may result in an increase in the crater diameter and therefore may increase the N-factor so long as the previously mentioned nonlinearities in ablation efficiency are absent. As another example, the crater diameter also depends on the distance to the target surface, so the closer the fiber is to the target stone, the smaller the crater diameter will be and therefore a smaller N-factor will result. In addition, increasing the fiber speed ν leads to a lower N-factor.

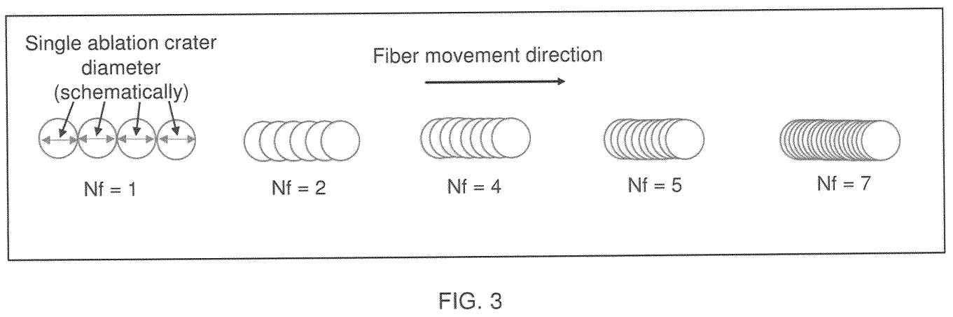

Visualization of the N-factor can be represented as in the schematic shown in FIG. 3. As shown in FIG. 3, in accordance with certain aspects Nf can be considered to correspond to a number of laser pulses delivered to a treatment zone having a diameter approximately equal to a diameter of a laser crater (D_{c}) created by pulsed laser energy that is directed at the treatment zone on a target (e.g., calculi). For instance, as indicated in FIG. 3, N_{f}=2 means that the fiber scans the target surface (e.g., during dusting) any point within this scan is exposed to two consecutive laser pulses. In other words, the scanned region received a total of two laser pulses at every point along the scan. Likewise, N_{f}=4 means that the fiber scans the target surface such that each spot within the scan is exposed to four consecutive laser pulses.

Considering expressions (4) and (7), the ablation efficiency can be expressed in terms of N_{f} as:

$${A}_{E} = ( S * f * {D}_{c} ) / ( \overline{P} * Nf ) \qquad ( 8 )$$

Note that S*D_{c} is proportional to the crater volume after the cumulative effect of N_{f} pulses and therefore S=S(N_{f}) and D_{c}=D_{c} (N_{f}). As will be appreciated, the maximum ablation efficiency corresponds to the first pulse delivered to treatment spot, N_{f}=1, because, despite the increase in multi-shot crater volume with an increase in N_{f}, the crater size does not increase proportionately. For example, the crater size after two pulses is less than double the single-pulse crater size. Therefore, the ablation efficiency decreases with each succeeding laser pulse. Nevertheless, a dusting ablation technique assumes that the fiber is scanning with continuous groove formation and not the fabrication of separate drills/holes/craters.

As will be shown below, in fact, the ablation crater diameters will “saturate” or reach a constant value asymptotically, as N_{f} increases. This means that at some moment in time, additional pulses (an increase of the N-factor) do not provide increasing crater diameter and depth and, therefore, volume. The ablation efficiency of each pulse after saturation onset is zero and, therefore, the total ablation efficiency begins to decrease rapidly.

There is a point at which an N-factor value provides continuous target scan/groove on the one hand and provides sufficient ablation efficiency of each subsequent laser pulse on the other hand. Indeed, it may be asked: “What purpose does an extra exposure to the current target spot by an extra pulse serve, if it does not lead to any visible/worthy ablation?” This point is underscored with the philosophy that reducing energy deposited into the body as much as possible is a desirable objective. In accordance with certain aspects, it is better to target/move the fiber into a new spot/location so as not to waste the energy and time on merely heating the water in the surrounding area. As such, a simple increase in the pulse energy and/or pulse frequency is not an optimal way (and is sometimes even detrimental) to increase the ablation efficiency, as is the conventional approach taken by many doctors.

Another aspect of implementing the N-factor is to adopt the most efficient laser parameters or set of laser parameters (that provide maximum ablation efficiency with minimal energy) to a doctor's typical speed of movement. While the speed of the fiber is a poorly controlled parameter that is fully dependent on clinician technique, N_{f} will also be a continuously changing factor. Still, a laser configured with a smart assistant mode (implemented by a controller) is capable of using feedback to control (in real time or by preset) a laser parameter (e.g. pulse energy, pulse frequency, average power, pulse width, peak power etc.) to maintain an efficient procedure.

In general, the greater the pulse energy, the larger the crater size, and the higher the fiber speed the doctor can use to provide efficient ablation. Alternatively, the doctor could use lower pulse frequency which has a similar effect on the N-factor and ablation efficiency as an increase in fiber speed (N_{f}=f D_{c}/ν). On the other hand, if a doctor tends to use low fiber speed, the pulse energy and/or pulse frequency should be limited as well, in order to be effectively adopted to a doctor's style of ablation process (A_{E}=S v/(Eₚ f).

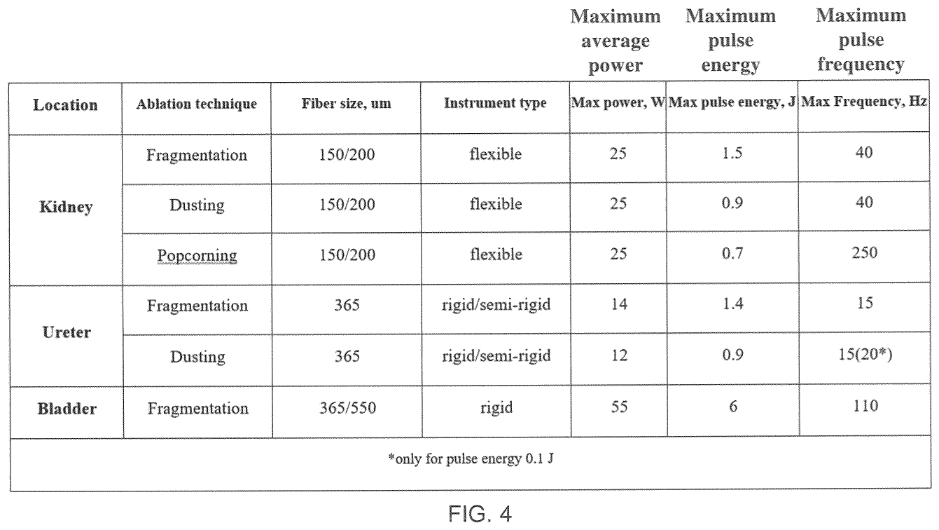

There is also an important aspect of the N-factor limitation related to the basic safety of the procedure. To avoid overheating and damage to organs, there are basic upper limits (or ranges), referred to herein as safety parameters, for applicable laser operating parameters, such as pulse energies and average powers, that are based on safety considerations. These safety parameter limits (or ranges) depend on the organ type. FIG. 4 is a table showing limits on various safety parameters for different organs. Using expression (7), the N-factor may be considered to depend at least in part on both pulse energy and pulse frequency, which in turn, depend on the above safety considerations that must first be reviewed and authorized, before implementing any laser operating parameter presets.

Despite the fact that laser lithotripsy has numerous advantages over other methods of choice for removing stones e.g., mechanical, acoustic/ultrasound, etc., there still exist various issues and challenges that need to be addressed including increasing procedure safety, shortening the time of procedure, maximizing the efficiency of the procedure, etc. The many factors described above directly influence these challenges and need to be considered altogether.

Thus, it would be of great value to have an intelligent laser system (a so-called smart assistant mode) that can automatically calculate, determine, and suggest an optimal set of laser parameters or optimal parameter ranges (such as pulse energy, frequency, peak power, pulse width, etc.) for laser lithotripsy in real time. Such an algorithm depends on the physics/nature of the ablation process and at least some of the pertinent varying factors (fiber diameter, average power, organ type, endoscope type, doctor's typical fiber speed, etc.) in order to increase the efficiency of the procedure and shorten the procedure time. The specific purpose in this case is an algorithm that adjusts laser parameters in order to continuously optimize the N-factor (e.g., ensure that Nf is less than or equal to Nfmax), as described in more detail below.

### Experiments, Methods, and Measurements

In accordance with various embodiments, an implemented smart assistant mode is provided to help a doctor choose the exact laser mode (i.e., a set of laser parameters) which is most efficient to ablate a current target (e.g., stone or soft tissue). As used herein, such treatment is calculi or stone (hard tissue) treatment, and as used herein, the term “calculi” refers to calculi (stones) present in anatomical locations, such as the ureter, kidney, or bladder. Calculi includes all types of stones in a human or animal body. However, it is to be appreciated that a similar methodology can also be applied to soft tissue ablation, cutting, incision, and excision. The choice of which laser mode should be done prior to the procedure or during the procedure in a real-time manner. In order to accomplish this, the ablation efficiency of the stone and the procedure needs to be characterized, and an understanding needs to be achieved of what the ablation efficiency is dependent upon and, therefore, what factors are most important to increase the ablation efficiency.

As was mentioned above, the ablation efficiency depends on many dynamic and static variables/parameters/factors. Despite the fact that some of these variables are static or not precisely controlled (e.g., speed of fiber movement, fiber size), other available parameters can be precisely controlled in real time during the procedure (e.g., laser settings such as pulse energy, pulse frequency, peak and average power, etc.).

The point is to understand the relationship between these different classes of factors and how they influence the ablation efficiency. This understanding is the key to an optimal smart assistant mode implementation.

There are a few ways to determine these relationships. A first approach is to study a theoretical model (scientific theory) of the processes that occur during laser ablation of the stone/tissue and identify and define the variables based on this theoretical model. However, it has been frequently found that predictions of a theoretical model can differ from actual measurements because there are important factors/variables not included in the theoretical model. However, even when the exact representation is known and the theoretical model is incomplete, an analytical approximation may yield a sufficiently accurate solution for the problem while significantly reducing its complexity.

A second approach is to perform a heuristic, analytical approximation that comprises the following: a comprehensive experimental program is undertaken that includes controllable and uncontrollable factors during the procedure, provides appropriate measurements, analysis of the results, and development of an analytical, approximate model of the ongoing ablation process that predicts to a certain level of accuracy the experimental results. This model can be used to calculate and predict ongoing ablation processes and therefore make a choice of appropriate settings of laser parameters that provide maximum ablation efficiency in clinical case.

As an example of this approach, Applicant has identified and characterized parameters relevant to the objective of improving safety and efficiency of surgical procedures for laser lithotripsy and soft tissue manipulation and has introduced an analytical model and derived expressions (5) and (8), above, for the lasing time and procedure length that depend on ablation efficiency which, in turn, depends on laser parameters and the N-factor. As the next step in such a program, Applicant has carried out an extensive, comprehensive in-vitro series of experiments of laser ablation in scanning modes on the surface of a stone, to be discussed below, which data will provide determination of the optimal N-factor that, in turn, defines the most efficient use of the laser for a given clinical procedure.

Bego stone was used as the targeted stone in these experiments. This stone is comprised of a special, heavy-duty plaster and is commonly recognized in the industry as a good model of a uric phantom stone. The Bego stones had dimensions of about 60×40×5 mm, and had both even and flat surfaces, and were placed in water inside a cuvette. The stone surface level was controlled by a level tool. A surgical fiber tip with a factory cleave was directed at the stone surface at a right angle and a predetermined, precisely controlled distance (gap). A Thulium fiber laser was used to produce directed optical pulsed energy. A motorized precision linear 2-axis stage was used to move the fiber to a new location after each impact of laser pulses (drilling or scanning) to form an array of laser-treated sites. Scanning of the fiber at an adjustable, constant speed parallel to the stone surface was controlled by a motorized X-Y linear stage. The first objective of the experimental program was to identify and quantify laser-produced craters as functions of laser parameters in the stone model.

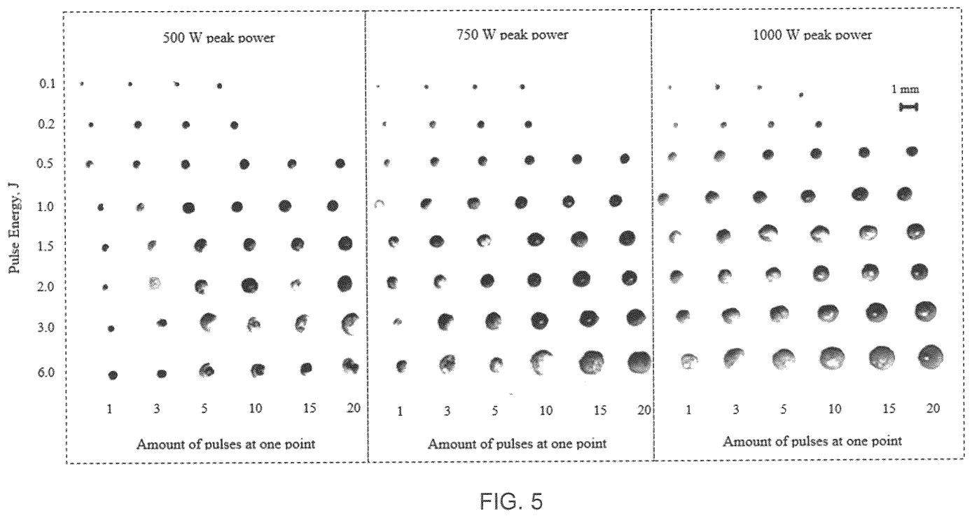

A matrix/array of craters was formed on the stone surface, with each crater created by specific laser parameters, the range of which is defined below:
1. The surgical fibers used had a core diameter in a range of 0.2-0.94 mm.
2. The distance between the fiber tip and the stone surface had a range of 0.1 (quasi-contact)-1 mm.
3. Pulse energy was in a range of 0.1-6 J.
4. Peak power was in a range of 250-2000 W.
5. The number of pulses in one point (N-factor) was in range of 1-20 per crater (pc) FIG. 5 shows photographs of an example of a crater array on the surface of Bego stone after the impact of laser pulses as a function of pulse energy (E) and N-factor (N_{f}) for 500, 750, and 1000 W peak powers (Pₚₑₐₖ) (all using a 200 μm fiber and a 0.1 mm gap between the fiber and Bego stone surface).

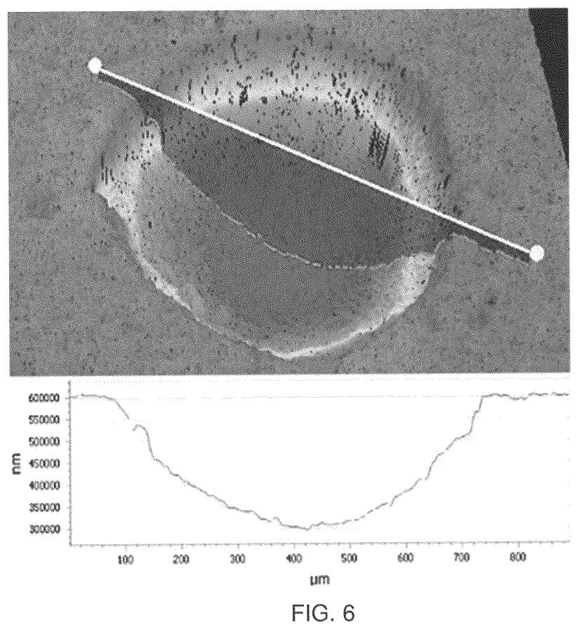

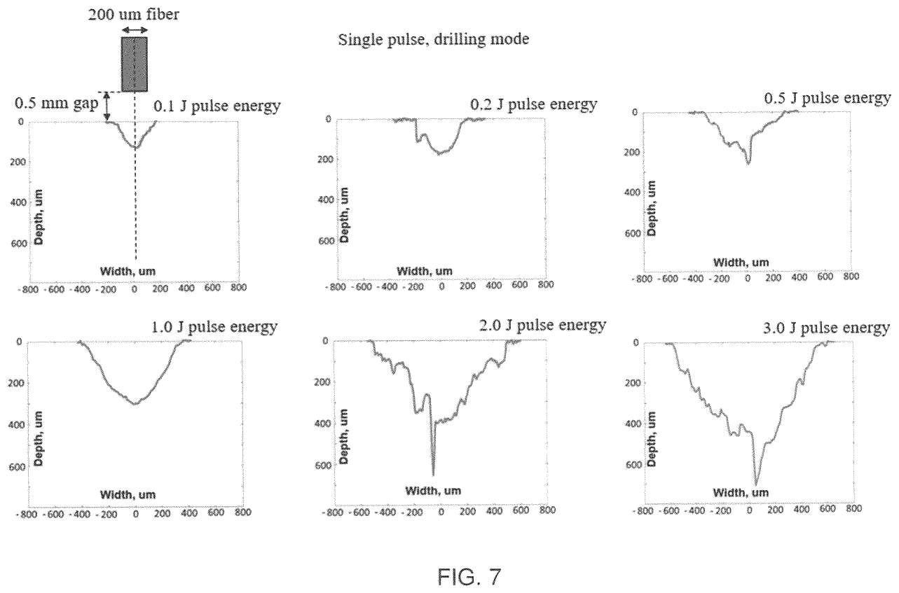

After drying the Bego stone (waiting for 24 hours at 25° C. prior to measurements), all crater diameters and cross-sections were measured with an optical profilometer (Zygo NewView™ 8300), as exemplified in the diagram shown in FIG. 6. The cross-section of each crater has a “U-shape,” as shown in the graphical results shown in FIG. 7 (and as also seen in FIG. 6) which shows the “U-shaped” depth profiles of different craters as a function of pulse energy (single pulse, 500 W peak power). The crater diameters were determined from the digitized images utilizing sampling with 886 points per crater (pc). The crater's size and shape (diameter, depth, cross-section, volume) relates to or is otherwise a function of the laser pulse parameters (pulse energy, N-factor, peak power) and other conditions (such as fiber diameter and gap).

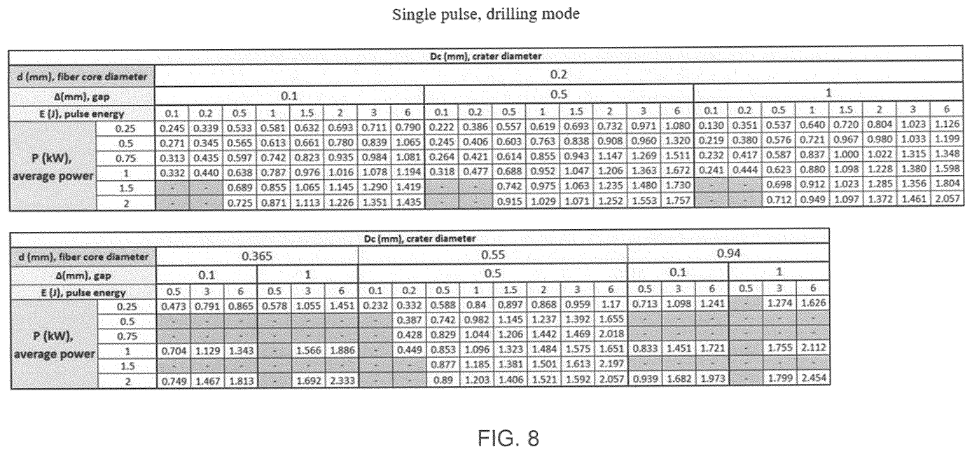

FIG. 8 is a table of the diameter of craters formed by a single laser pulse. The results indicate that the crater diameter increases with increasing pulse energy or peak power due to the increase in the laser total fluence or the laser fluence rate (power density), respectively. Similarly, the crater diameter increases with an increase in the fiber diameter for higher powers but not for lower power because the lower power pulses are closer to the threshold power density and beam profile becomes important. With increase in gap the trends are complicated by the interactions between laser, stone and water due to the Moses channel dynamics.

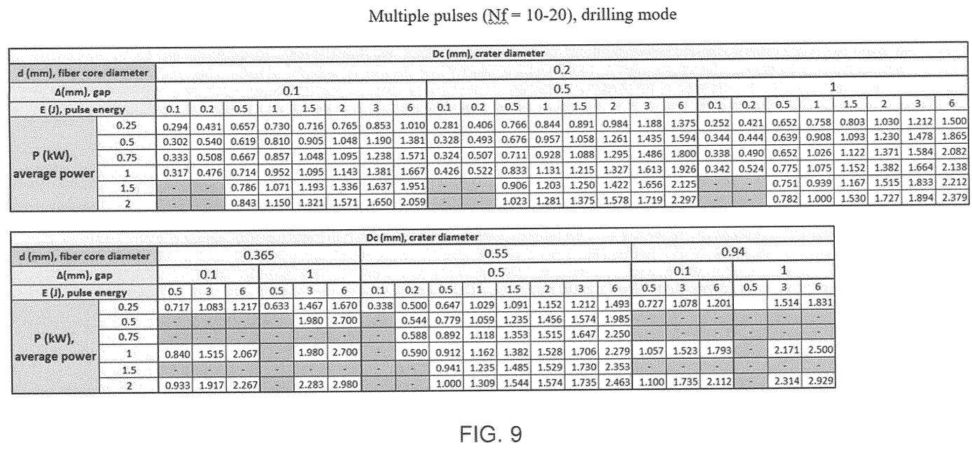

The crater diameter exhibits the onset of saturation when the N-factor value is in the range of 10-20, depending on other pulse parameters. This means that the rate of increase of the crater diameter with additional laser pulses significantly decreases. FIG. 9 shows the results of saturated crater diameters after 10-20 pulses (N_{f}=10-20). The data indicates that ‘saturated’(maximum) crater diameters exhibit similar trends as those exhibited by the diameters of the single-pulse craters with varying pulse energy and peak power for given fiber diameters and gaps. However, the value of the diameters for the saturated craters is not even twice that of single pulse craters.

The matrix/array of scans/grooves is dependent on varying/changing parameters. The experiments utilized the following ranges of factors (note that the distance between the distal tip of the fiber and the stone surface was kept constant at 0.5 mm for all data points):
1. The surgical fibers had core diameters in a range of 0.2-0.94 mm.
2. Pulse energies were in a range of 0.2-6 J.
3. Peak powers were in a range of 250-1000 W.
4. Average powers were in a range of 10-40 W.
5. Pulse frequencies were in a range of 5-55 Hz.
6. The speed of fiber scanning was constant for each scan and in a range of 0.5-10 mm/s.
7. N_{f}, N-factor was in a range of 1-20

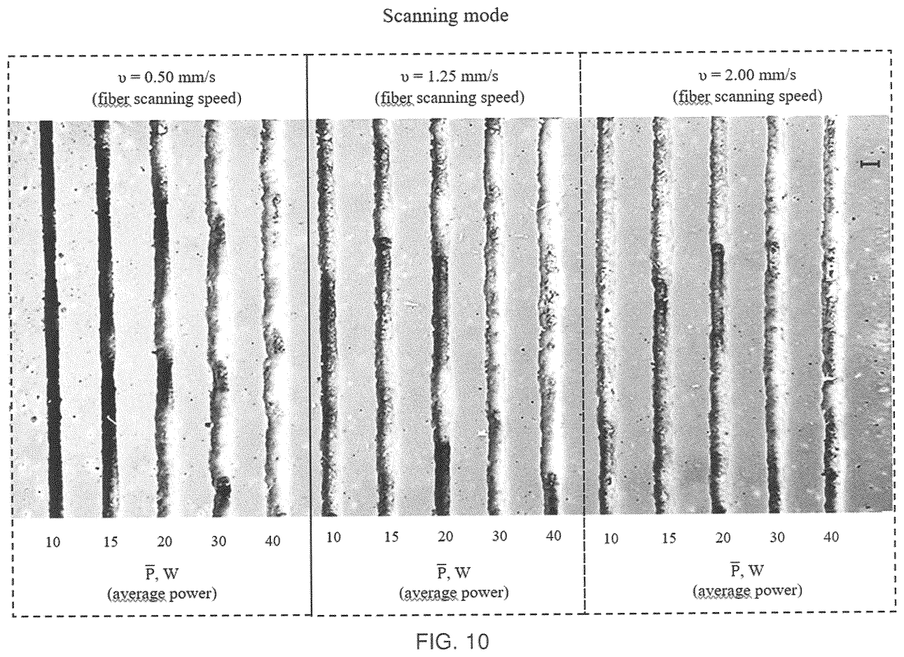

Each scan was unique to itself. As an example, FIG. 10 shows photographs of groove arrays on the surface of the Bego stone after the impact of scanning laser pulses as a function of average power (P) for 0.5, 1.25, and 2 mm/s fiber scanning speed (200 μm fiber, 500 W peak power, 0.8 J pulse energy, 0.5 mm gap between fiber and Bego stone surface). Therefore the range of number of shots per point, N_{f}, across the grooves in this figure is 4-64 shots/spot.

The total collected data points with which to measure scan depths and cross-sections is 88 pc (per crater). Each scan's depth and cross-section was measured with the same optical profilometer.

After the scanning experiments, the area of the ablation crater cross-section in the vertical plane (S, mm²) was measured for each of the different laser parameters: pulse energy (E, J), average power (P̅, W), pulse frequency (f, Hz) and fiber scanning speed (ν, mm/s).

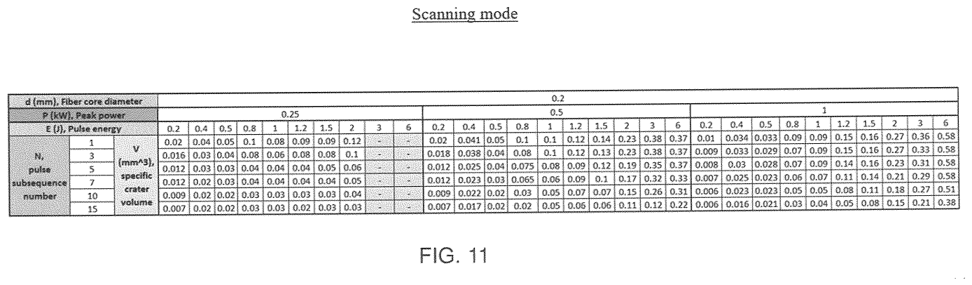

FIG. 11 is a table showing the results of groove measurements after impact from a scanning pulse and indicates the calculated specific crater (groove) volume (the volume contribution of an individual pulse) in scanning mode for a 0.2 mm fiber core diameter as a function of peak power, pulse energy, and N (pulse sequence number). The specific crater (groove) volume (in mm³) was calculated according to the following expression:

$$V = ( S * υ ) / f \qquad ( 9 )$$

where
S (mm²)=area of the crater (groove) cross-section,
ν (mm/s)=speed of fiber scanning (constant during each scan), and
f (1/s)=pulse frequency.

### Calculation, Approximation, and Discussion

An analytical expression with adjustable coefficients was fit to the experimental data to describe the relationship between the diameter of the crater at a single point after the first laser pulse (FP). The result is the formula:

$${D}_{c}^{FP} = ( 2 NA Δ + d ) ( 0.12 \sqrt[4]{{P}_{peak}} ln ( {E}_{1} / ( 0.25 π {{F}^{′} ( 2 NA Δ + d )}^{2} ) ) - \sqrt{{P}_{peak}} / 250 ) ++ ( 1.82 d ) ( 0.21 - 0.0 \; 25 {E}_{1} ) \qquad ( 10 )$$

where
NA=fiber numerical aperture,
Δ(mm)=gap between fiber tip and stone surface,
d (mm)=fiber core diameter,
Pₚₑₐₖ (W)=peak power of laser pulse,
E₁ (J)=single pulse energy,
F′(J/cm²)=a threshold of targeted stone destruction which is 20 J/cm² for Bego stone model at 1.94 μm wavelength (threshold ablation).

Formula (10) is an analytical approximation that is dependent on physical factors. For example, the factor (2NAΔ+d) is the diameter of the irradiated spot on the target as a function of the distance to the target, Δ, the fiber diameter (core), d, and numerical aperture NA. The factor E₁/(0.25 T (2NAΔ+d) 2) can be described in units of J/cm² and reflects the energy fluence (energy density) on the target surface. Dividing this factor by F′, one can obtain the fractional excess of energy/fluence exceeding the ablation threshold. The remaining expressions/values are whole correction factors having minor physical meaning.

Formula (10) is not the only possible expression that approximates and describes or otherwise reveals the crater diameter behavior as a function of the variables of interest. Many other analytical approximations can be found based on experimental data, but the solutions for the crater diameter should be equal to one another no matter which formula is used because they must all fit/predict the same experimental data.

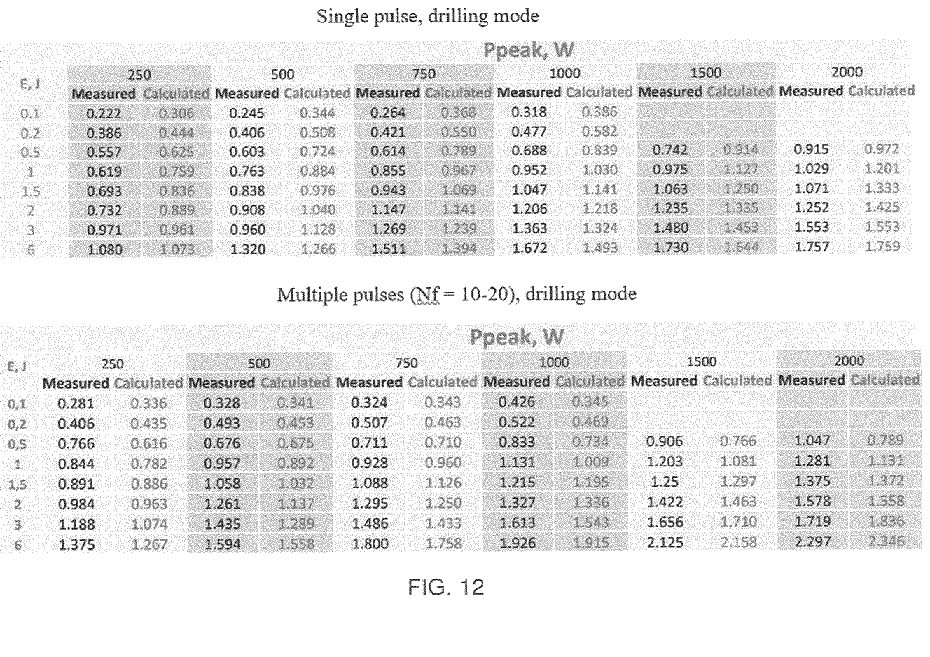

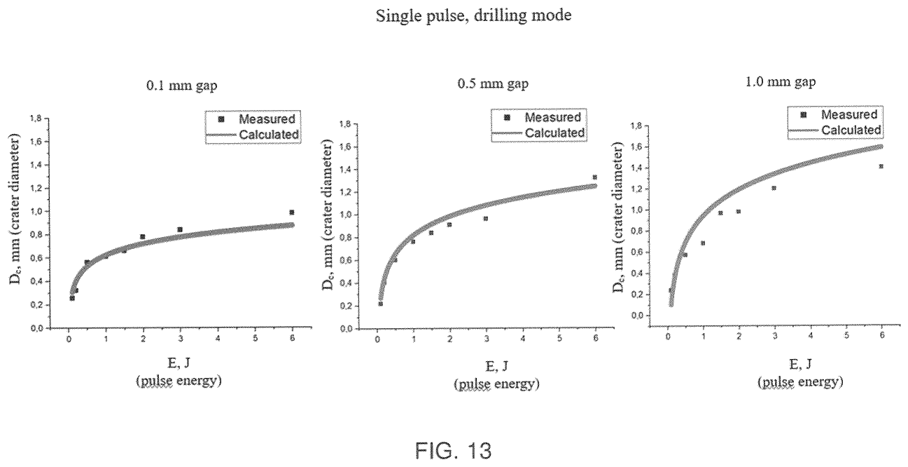

FIG. 12 is a pair of tables showing a comparison of experimental and calculated data using the proposed formula (10) for the crater diameter after impact from single and multiple pulses for 200 μm fiber and 0.5 mm gap as a function of pulse energy and peak power. FIG. 13 shows an example of the application of formula (10) (solid line in graphs of FIG. 13) and reflects graphs showing a comparison between measured experimental data and the calculated data (via proposed formula 10) for 0.2 mm fiber and 500 W peak power for gaps of 0.1 mm, 0.5 mm, and 1 mm. The shape of the curves shows that the relationship between the crater diameter and the pulse energy is logarithmic. Proposed formula's (10) error (i.e., the difference between measured and calculated data divided by the average of the measured and calculated data) is within 30% for all energies.

Another analytical approximation formula was found to describe the existing relationship between a saturated crater diameter after several laser pulses (Nf=10-20):

D_{c}^{SAT}=(2NAΔ+d)(1/(1.61+10.23d⁴+(0.92−2.97d)Δ))×(1+(\sqrt{Pₚₑₐₖ}/12.85)1n((12.85//\sqrt{Pₚₑₐₖ})(1/(6.4−6.75d))(E/(0.25πF′(2NAΔ+d)2−1)+1)) (11)

Again, the Formula (11) is also not the only possible expression capable of predicting saturated crater diameter as a function of the dependent variables. Many other analytical approximations can be found and fit to the experimental data, but all approximations should result in the experimental crater diameters.

In accordance with at least one embodiment, the crater diameter D_{c} is in a range of the expression in Eq (10) and the expression in Eq (11), according to the following expression:

$${D}_{c}^{FP} \underline{<} {D}_{c} \underline{<} {D}_{c}^{SAT}$$

$$or$$

$$( 2 NA Δ + d ) ( 0.12 \sqrt[4]{{P}_{peak}} ln ( {E}_{1} / ( 0.25 π {{F}^{′} ( 2 NA Δ + d )}^{2} ) ) - \sqrt{{P}_{peak}} / 250 ) + ( 1.82 d ) ( 0.21 - 0.0 \; 25 {E}_{1} ) \leq {D}_{c} \leq ( 2 NA Δ + d ) ( 1 / ( 1.61 + 10.23 {d}^{4} + ( 0.92 - 2.97 d ) Δ ) ) \times ( 1 + ( \sqrt{{P}_{peak}} / 12.85 ) ln ( ( 12.85 / \sqrt{{P}_{peak}} ) ( 1 / ( 6.4 - 6.75 d ) ) ( E / ( 0.25 π {{F}^{′} ( 2 NA Δ + d )}^{2} - 1 ) + 1 ) ) ,$$

where the variables are defined above for those equations.

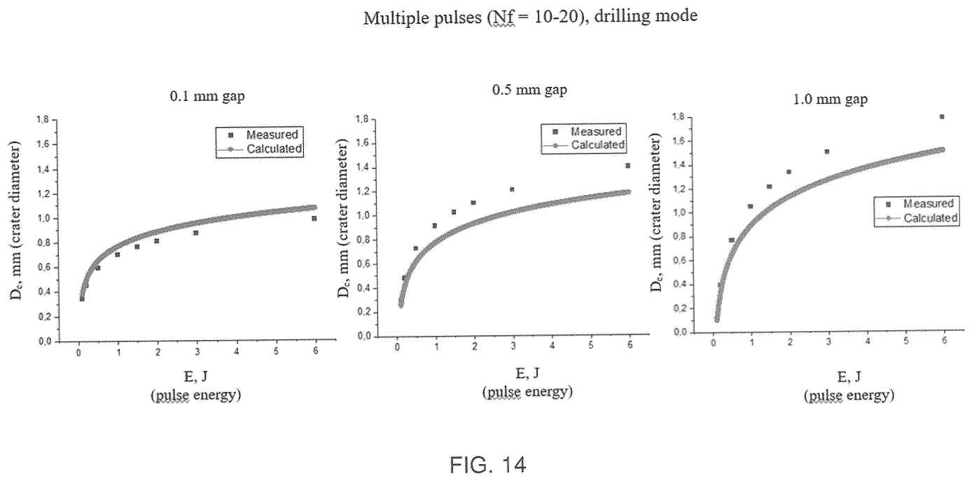

FIG. 14 shows three graphs from which one may obtain the saturated crater diameter using the application of Formula (11) and reflects a comparison between the measured experimental data and the calculated data (formula (11), for a 0.2 mm fiber and 500 W peak power for gaps of 0.1 mm, 0.5 mm, and 1 mm. As with FIG. 13, the shape of the curves indicate that the relationship between the crater diameter and the pulse energy is also logarithmic. Proposed formula's (11) error (i.e., the difference between measured and calculated data divided by the average of the measured and calculated data) is within 30% for all pulse energies.

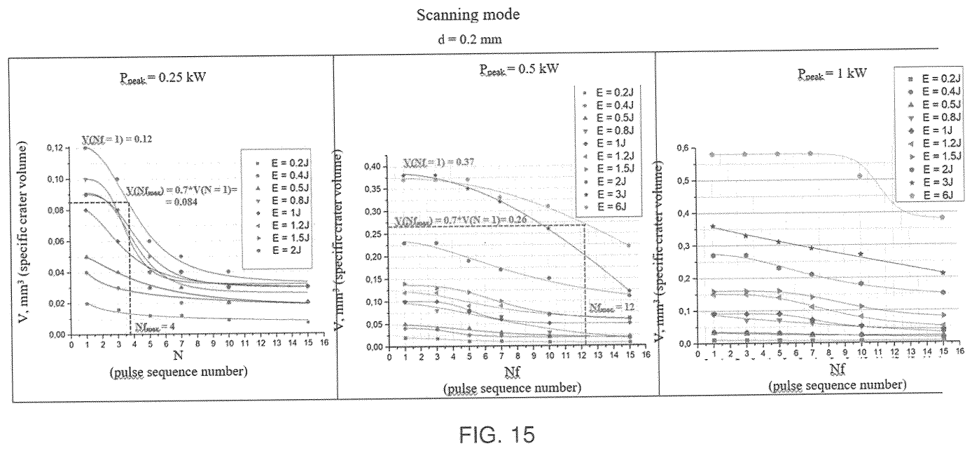

FIG. 15 shows in three graphs of calculated specific crater volumes for 0.2 mm fiber for different N_{f} (pulse sequence number) values as a function of peak power and pulse energy. The gap between the fiber tip and the stone is 0.5 mm. The dots (data points) reflect experimental data points and the lines reflect the curves of calculated data approximations. The error is less than 30%.

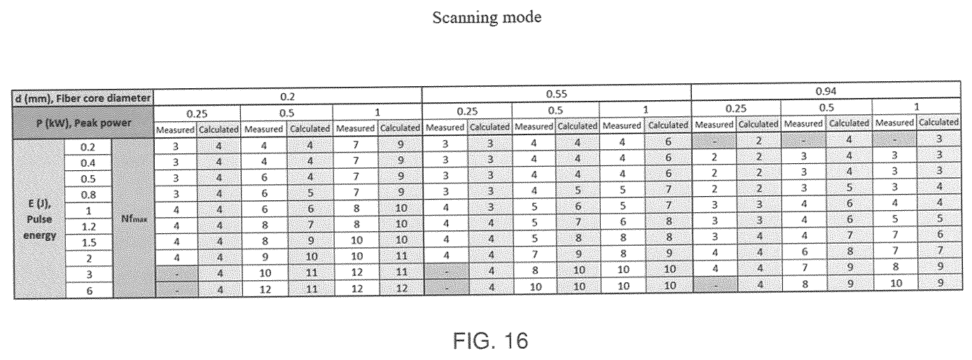

FIG. 16 is a table showing a comparison between the measured and the calculated most efficient values of the number of pulses in one spot for different fiber diameters as a function of peak power and pulse energy. The error is less than 30%.

As was stated previously, increasing the pulse frequency while using the same pulse energy and increasing Nf (the number of pulses into one spot) does not necessarily lead to an increase in ablation efficiency or in overall efficiency of the procedure. It is clear that starting from an “exact moment of time,” each further irradiating pulse on the same target spot does not necessarily lead to any efficient ablation of the targeted volume. In accordance with various aspects, a new parameter can be named and utilized—i.e., the most efficient number of pulses in one spot, or Nfmax that satisfies the following equation:

$${A}_{E} ( {Nf}_{max} ) / {A}_{E} ( Nf = 1 ) = K \qquad ( 12 )$$

where A_{E}(Nfₘₐₓ) is the average ablation efficiency per pulse after Nfₘₐₓ pulses in a single spot, and A_{E}(N_{f}=1) is the ablation efficiency of the first pulse to the spot which is the maximum possible per-pulse ablation efficiency (or ablation efficiency of single pulse). The K value is a predetermined value, and in accordance with some embodiments can be selected to be in the range of about 0.25-0.75 (25-75%) (inclusive), more preferable about 0.50-0.75 (50-75%) (inclusive). In some embodiments, the K value is in a range of about 25-75% inclusive, and in some embodiments the K value is in a range of about 25-50% inclusive. Higher K values provide higher ablation efficiencies and shorter procedure times. In accordance with at least one embodiment, Nfmax is in a range of 1-12 inclusive. In another embodiment, Nfₘₐₓ is in a range of 1-7 inclusive. In yet another embodiment, Nfmax is in a range of 1-5 inclusive, and in yet another embodiment Nfmax is in a range of 1-3 inclusive.

Expression (12) implies that Nfₘₐₓ is determined such that it is the exact number of pulses in one spot that provides ablation efficiency not less than K value that corresponds with a maximum possible ablation efficiency or of the ablation efficiency for a single pulse. In some embodiments, Nfmax corresponds to a number of laser pulses delivered to the treatment zone having a diameter approximately equal to the laser crater diameter D_{c} that provides an average calculi ablation efficiency per pulse for a maximum number of pulses that is greater than or equal to the predetermined K value corresponding to an ablation efficiency achieved after impact from a single (first) pulse. In other words, every current spot of the target is irradiated by the least number of most efficient pulses. In one embodiment, when the ablation efficiency of an additional pulse reduces to K to a value below the maximum possible ablation efficiency or from the ablation efficiency of a single pulse, there is no sense in irradiating the current spot by additional laser pulses and it is much more efficient to move the fiber into a new target spot during target scanning (dusting technique). In other embodiments the same holds true for K not less than 0.25, not less than 0.50, or not less than 0.75 of maximum values.

Since ablation efficiency and ablated volume are bound together by expression (3), Nfₘₐₓ can also be found by using a specific crater volume:

$$V ( {Nf}_{max} ) / [ {Nf}_{max} * V ( Nf = 1 ) ] = K \qquad ( 13 )$$

Expression (13) means that Nfₘₐₓ can be determined as the exact number of pulses in one spot that provides per-pulse stone volume ablation not less than K fraction of the maximum possible ablation volume which is created with impact of the first pulse. In other words, every spot of the target is irradiated by the smallest number of the most efficient pulses. Although the example in Expression (13) provides per-pulse stone ablation volume not less than K of the maximum possible (first pulse) ablation volume, other percent values are also within the scope of this disclosure, including not less than K=0.25, not less than K=0.50, and, at most, not less than K=0.75 of the maximum possible first-pulse ablation volume. When the ablation volume of an additional pulse reduces to 25, 50, 70, or 75% of the maximum possible first-pulse ablation volume, there is no reason to irradiate this current spot by additional laser pulses and it is much more efficient to move the fiber into a new target spot during target scanning.

According to at least one embodiment, Nf is compared to Nfmax. This can be done by a controller, as discussed in further detail below. In response to a determination that Nf is less than or equal to Nfmax, then the laser source is controlled using one or more laser operating parameters that are used in calculating Nf to control the laser source during a laser lithotripsy procedure. In response to a determination that Nf is greater than Nfmax, then the controller (or a user) can adjust at least one laser operating parameter that is used to control the laser during a lithotripsy procedure. In this instance, the goal is to adjust the laser operating parameters such that the Nf value is equal to or less than Nfmax. This approach is discussed in further detail below. In addition, a controller may be configured to display on a display device at least one laser operating parameter based on the comparison, and in some instances may display on a display device at least one of Nf and Nfmax.

In accordance with at least one embodiment, Nf is selected or otherwise adjusted to be less than or equal to Nfmax. In some embodiments this is done by adjusting one or more laser operating parameters, such as the pulse energy, pulse frequency, average power, peak power, and/or pulse width. As note above, Nfmax corresponds to the number of laser pulses delivered to the tissue surface at a specific spot in the treatment zone and every pulse at that spot ablates a volume of stone (increases the diameter/depth/volume of the crater formed from the previous pulse) to provide an average per-pulse stone ablation efficiency that is in some embodiments not less than 25%, in some embodiments not less than 50% and in yet other embodiments is not less than 75% of the ablation efficiency achieved with the first (single) pulse. In one embodiment, the 75% criterion is calculated according to Formula (14) described below.

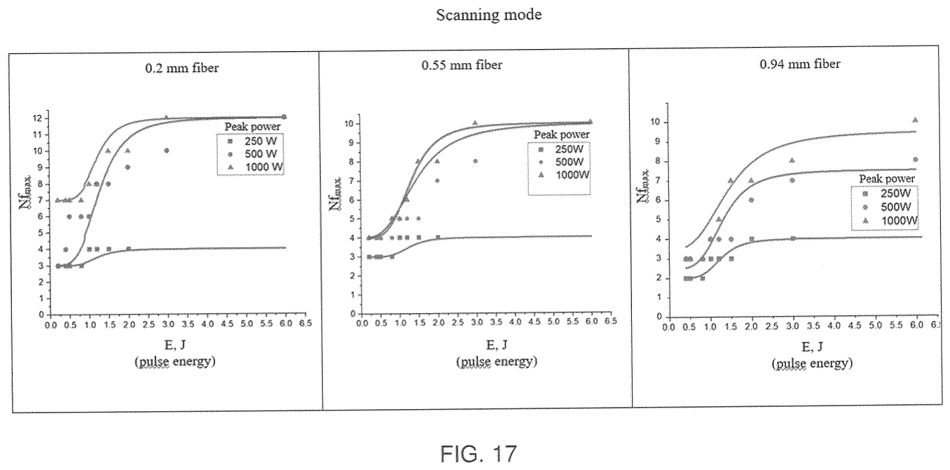

This approach is shown in the three graphs of FIG. 17 where one can see determined Nfmax (solid line) for each fiber diameter (0.2, 0.55, and 0.94 mm) for a peak power of 0.5 kW. The graphs show the most efficient N-factor as a function of pulse energy and peak power for different fiber diameters using a 0.5 mm gap between the fiber and the Bego stone surface. The data points reflect measured experimental data and the solid line curves indicate calculated data using the analytical approximation formula. For example, for a 0.2 mm fiber diameter with 0.5 kW peak power and a 6 J pulse energy, the maximum ablation volume (ablation volume for single pulse) is V (N_{f}=1)=0.37 mm³. Using expression (13), the calculated (V (Nfₘₐₓ)/(Nfₘₐₓ*V (N_{f}=1))=0.7=0.7*0.37 mm³=0.26 mm³. The corresponding number of pulses that correspond to the calculated ablation efficiency value: Nfmax=12, found by interpolating the volumes between Nfmax=10 and 12 in the table of FIG. 11. This value represents the maximum number of pulses in one spot with acceptably high per-pulse ablation efficiency.

FIG. 16 also shows this approach having the most efficient number of pulses in one spot for different fiber diameters (0.2, 0.55 and 0.94 mm) as a function of peak power (0.25-1 kW) and pulse energy (0.2-6 J).

Using measured data of Nfₘₐₓ from the table in FIG. 11, another approximate formula was determined that describes the dependence of the highest efficient N-factor on other factors, namely, fiber-stone gap, d, peak Power, and pulse energy, E and K=0.75:

$$Nfmax = ( 12.4 - 3.4 d ) + ( 3.3 d - 8.4 ) / ( 1 + 4.7 \times 1 {0}^{- 5} exp ( {P}_{peak} / 0.0375 ) ) - ( ( 14.7 d - 16 ) + ( 80 - 65 d ) {P}_{peak} + ( 55 d - 62 ) {P}_{peak}^{2} ) / ( 1 + 0.34 {E}^{5.5 - 2.6 ( Ppeak )} ) \qquad ( 14 )$$

Since the number of pulses is a natural number, the result of expression (14) must be rounded to the nearest integer. Formula (14) is also not the only possible expression that describes the most efficient N-factor behavior as a function of the variables. This formula is only one of several possible approximation formulas that describe this relationship. Thus, many other possible analytical approximations can be found based on gained experimental data, but the solution for the most efficient N-factor equation must predict the experimental data from FIG. 11.

In accordance with various embodiments, Nfmax is determined based on a database of stored Nf data obtained from experiments (e.g., the data behind FIG. 11). Pre-clinical and clinical trial data from laser lithotripsy procedures was analyzed and Nfmax values were calculated for different combinations of input and procedure parameters to determine the most efficient number of pulses in one spot and that also complied with safety parameter thresholds so that tissue damage or other harmful effects were not encountered.

Returning to FIG. 17, the 3 graphs show an example of an application of formula (14) and a comparison between measured experimental data and calculated data via the proposed formula for 0.2 mm, 0.55 mm, and 0.94 mm fibers, for 250 W, 500 W and 1000 W peak powers, using a 0.5 mm gap. The calculated data using this proposed formula is also shown in the table of FIG. 16. Proposed formula's (14) accuracy (i.e., the difference between measured and calculated data divided by the average of the measured and calculated data) is within 30% for all pulse energies.

Based on the data in Formula 14, Table 16 and FIG. 17, Nfmax can be in the range 3-12. Nfmax increases with energy and peak power. This defines the optimal value of Nfmax is less than or equal to 12, preferably less than or equal to 7, more preferably less than or equal to 5 and most preferable less than or equal to 3. In accordance with various aspects, during treatment N_{f} may be less than the values in the above interval.

It is clear that the most efficient number of pulses Nfmax increases with an increase in peak power. For a given power, Nfₘₐₓ starts to grow and eventually saturates also with increasing pulse energy. For example, increasing the pulse energy from 0.2 J to 3 J for a 0.55 μm fiber and 1 kW peak power, Nfₘₐₓ increases from 4 to 10, but a further increase of the pulse energy to 6 J does not lead to an additional increase in Nfₘₐₓ. Proposed formulas (10), (11), and (14) are sufficient for an algorithm that provides a pulse energy and frequency for an optimized treatment for stone removal (maximum efficiency with the least amount of energy), while accounting for the described changing factors (e.g. fiber diameter, gap, peak power).

### Proposed Smart Assistant Mode Approaches

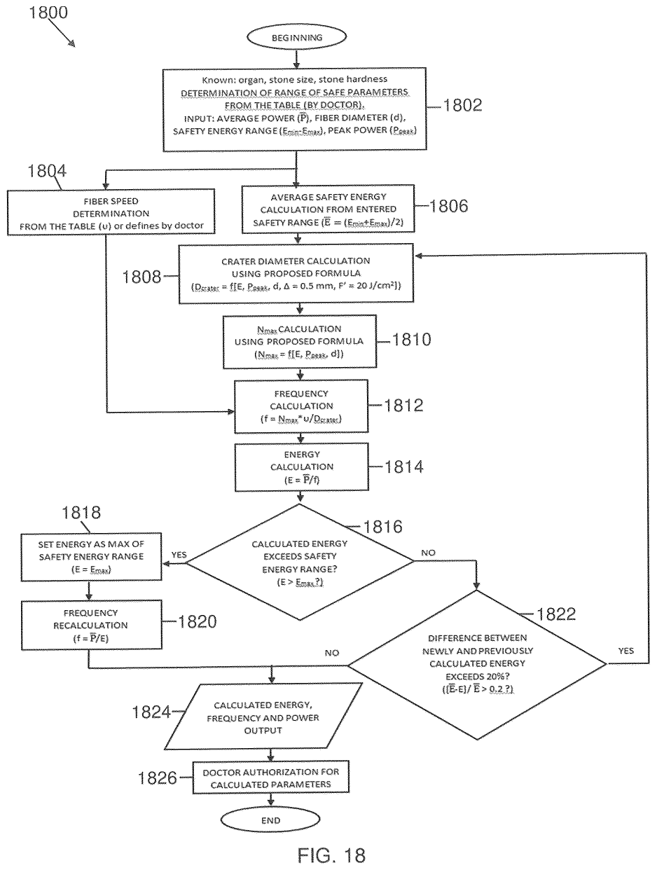

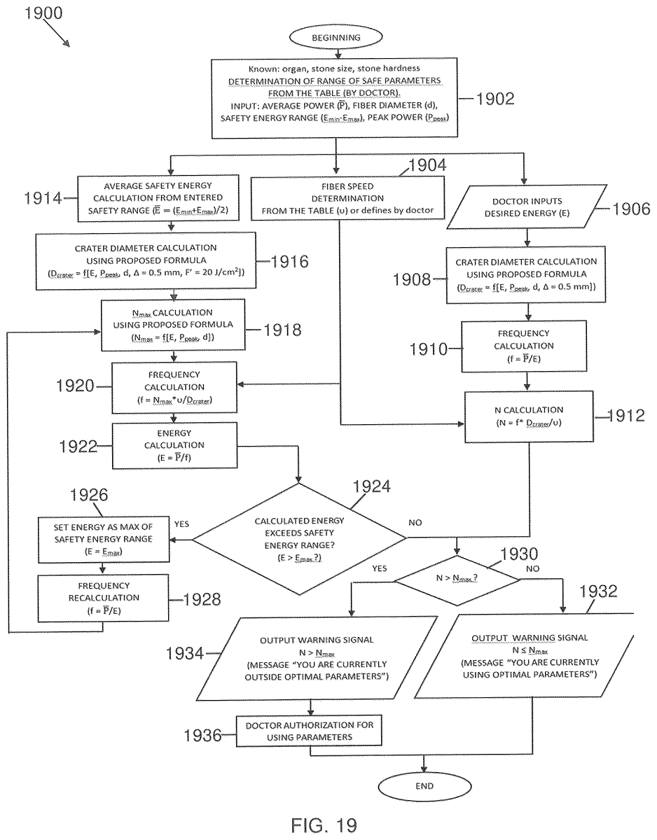

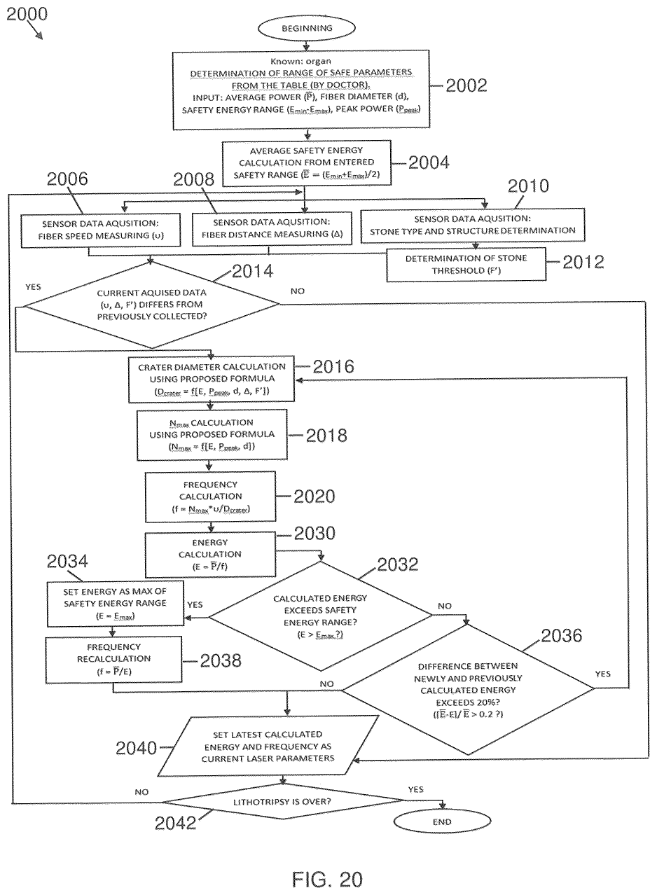

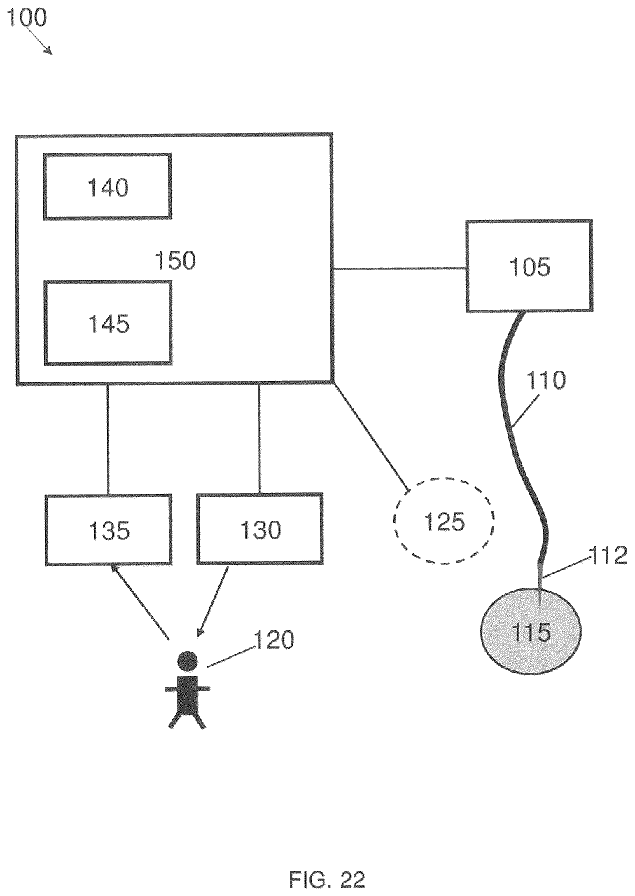

In accordance with various embodiments, there are a few ways to implement a smart assistant mode and its possible integration into a laser system. For the purpose of this disclosure, three non-limiting examples are discussed, a first as process 1800 in FIG. 18, a second as process 1900 in FIG. 19, and a third as process 2000 in FIG. 20. However, it is to be appreciated that other variations to the approaches are also within the scope of this disclosure. In addition, formulas (10), (11), and (14) can be used in other non-limiting approaches and applications of a smart assistant mode during laser lithotripsy. One or more steps included in these processes may be performed by controller 150 or components of the controller, which is discussed in further detail below regarding system 100 of FIG. 22. At the beginning of all proposed approaches, the first step (1802, 1902, 2002) comprises receiving or otherwise obtaining one or more input parameters. In accordance with various embodiments, the one or more input parameters comprise at least one of target parameters, system parameters, and safety parameters. The controller 150 may be configured to receive the one or more input parameters.

In some embodiments, the one or more input parameters comprise target parameters, such as information about the target and target location. The target parameters may comprise target type (e.g., calculi), target location (e.g., kidney, bladder, ureter), and/or one or more target characteristics. For example, according to an embodiment where the target is calculi, the one or more target characteristics may comprise size and hardness of the calculi. In addition, according to another embodiment where the target is calculi, the one or more target parameters may comprise a proposed ablation technique, such as fragmentation, dusting, or popcorning. These latter parameters may be based in part on one or more target characteristics, such as the calculi's size and hardness.

According to at least one embodiment, the one or more input parameters comprise system parameters, such as laser type, laser characteristics, and other information about the system that will perform the procedure. Non-limiting examples of system parameters include laser type, e.g., thulium fiber laser (TFL), Ho:YAG, Tm:YAG solid-state laser and/or laser wavelength(s), fiber diameter (i.e., the diameter of the optical fiber (e.g., optical fiber 110 in FIG. 22, also referred to herein as surgical fiber) that directs the laser energy at the target), and instrument type, such as flexible, rigid, and/or semi-rigid scopes.

In accordance with at least one embodiment, the one or more input parameters comprise safety parameters, including maximum laser operating parameters so that the patient undergoing the procedure is not exposed to unnecessary injury. In some embodiments, the safety parameters comprise at least one of maximum average power, maximum peak power, maximum pulse energy, and/or maximum pulse frequency. In some embodiments the safety parameters also comprise a minimum pulse energy.

The input parameters may be received or otherwise determined by the controller 150 a number of different ways. For example, a user 120 (e.g., a doctor) may input to system 100 via a user input device 135 (e.g., touchscreen) one or more input parameters. In one embodiment, the doctor 120 inputs at least one target parameter (e.g., target location, and if the target is calculi, the size and hardness of the target). In some instances, the target size and hardness may be determined by viewing the target beforehand using an imaging device. In another embodiment, user 120 (doctor) inputs to the system (e.g., via user input device 135 such as a touchscreen) input parameters that comprise one or more system parameters such as fiber diameter and/or a fiber numerical aperture. In another embodiment, user 120 (doctor) inputs input parameters that comprise one or more safety parameters such as: maximum average power, maximum pulse energy, and/or maximum peak power. In some embodiments, the user 120 may input one or more safety parameters such as maximum average power and a maximum pulse energy based at least in part on safety considerations using (for example) the table in FIG. 4. In other embodiments, the controller 150 itself may be configured to determine one or more safety parameters based at least in part on one or more other input parameters (e.g., target type, target characteristics, target location, fiber size, and/or instrument type). For instance, stored information (e.g., the table including information from FIG. 4) by the controller 150 may be used at least in part by the controller 150 to determine one or more safety parameters based on one or more other input parameters.

In accordance with another embodiment, controller 150 calculates and/or uses stored input parameters. For instance, in one embodiment, user 120 chooses (for example, using the user input device 135 such as a GUI touchscreen) proposed (predetermined) one or more input parameter values and controller 150 automatically determines input parameters such as maximum average power and the safety energy range (Emin-Emax) based on other input parameters, such as the organ being treated, (e.g. kidney, ureter, bladder), instrument type (rigid, semi-rigid, flexible), fiber diameter, and required ablation technique (fragmentation, dusting, popcorning).

In some embodiments, the fiber diameter can also be automatically determined by the controller 150, for example by using a built-in NFC chip or RFID chip (e.g., a sensor, such as sensor 125 in FIG. 22) associated with the surgical fiber instrument (e.g., endoscope).

In some embodiments one or more procedure parameters (also referred to herein as treatment parameters), such as laser operating parameters, can be automatically determined (calculated) by the controller 150. For instance, peak power may be automatically determined by controller 150 based on the potential/capacity/characteristics of the laser source 105 and safety parameters that are to be used for the procedure.

In accordance with various aspects, three examples of laser systems that utilize the N-factor as a treatment parameter are described below. In addition, other treatment parameters are utilized such as pulse energy, frequency, pulsewidth (or peak power) and average power to provide maximal ablation efficiency and minimal procedure time. For example one or two users (urologist or his assistant) select treatment parameters to keep the N-factor in an optimal range<N_{fmax} Example 3 of the laser system includes real time measuring of speed of the fiber and automatic adjustment of laser parameters to keep the N-factor in optimal range≤N_{fmax}.

### First Example of a Laser System with Smart Assistant Mode

A flowchart of an example of steps used in a first approach method or process 1800 using the smart assistant mode is shown in FIG. 18. As noted previously, one or more of the steps included in process 1800 may be carried out by a controller (e.g., controller 150 discussed below). An initial step at 1802 includes receiving (e.g., by the controller) one or more input parameters, as described above. According to at least one embodiment, the input parameters comprise target parameters. In one embodiment, the target parameters comprise target type, target location, and one or more target characteristics. In some embodiments the target type is a calculi and the target location is the kidney, bladder, or ureter. In certain embodiments the target type is calculi and the one or more target characteristics comprise size and/or hardness of the calculi. In some embodiments, the input parameters include at least one system parameter and in one embodiment the at least one system parameter comprises at least one of a fiber diameter, a fiber numerical aperture, and an instrument type. For instance, as shown in FIG. 22, an optical fiber 110 is used to direct pulsed laser energy, and the fiber diameter and fiber numerical aperture relate to the distal end (the end where the pulsed laser energy is emitted from the fiber) of this optical fiber 110. In some embodiments, the instrument type is a flexible or a rigid scope type. In another embodiment, the input parameters include at least one safety parameter, and in one embodiment the at least one safety parameter comprises maximum average power, maximum peak power, maximum pulse energy, and/or maximum pulse frequency.

In accordance with another embodiment, at least one procedure parameter (also referred to herein as process parameters) is determined. Procedure parameters may include one or more parameters used or otherwise implemented by the doctor and/or system when performing the lithotripsy procedure. Non-limiting examples of procedure parameters include pulse frequency, peak power, pulse energy, laser crater diameter, fiber speed, a gap between the fiber tip and target, and a target ablation threshold. In accordance with certain aspects, the N-factor N_{f} or maximum N-factor Nfmax, may also be considered to be procedure parameters and/or laser operating parameters.

In some embodiments, the at least one procedure parameter that is determined comprises the fiber speed (ν). This can be done automatically by the controller 150 using the table in FIG. 2 based on one or more previously received parameters (e.g., input parameters such as the organ type, instrument type, fiber diameter, etc.). Alternatively, the doctor can input the fiber speed that he or she requires or intends to use. Another approach is that the doctor chooses the proposed relative regimes/styles of fiber manipulation (e.g. slow, average, or fast) that he or she requires or intends to use and the laser system automatically determines the corresponding fiber speed (minimum, typical, maximum) using, for example, proposed data as shown in the table of FIG. 2. Speed of fiber movement in FIG. 2 was determined based on processing of endoscopic video captured during stone laser treatments performed by several different urologists. A particular value in this table can be averaged across different urologists or can be collected for a particular user and stored in the controller for an Nfₘₐₓ calculation. In other embodiments the doctor can define the fiber speed beforehand.

In accordance with some embodiments, an average safety pulse energy is calculated or otherwise determined based on one or more safety parameters. For example, in step 1806, an average safety pulse energy Ē is calculated based on a maximum pulse energy Emax and a minimum pulse energy Emin (e.g., a minimum pulse energy needed to perform ablation) and according to the following expression: (Ē=(Eₘᵢₙ+Eₘₐₓ)/2). Emin may be determined from stored data obtained from previous pre-clinical and/or clinical trial data and corresponds to a minimum pulse energy that provides a minimally acceptable efficiency (i.e., acceptable amount of ablation on a target calculi).

In some embodiments, at least one procedure parameter that is determined is the crater diameter Dc. For example, at step 1808 the controller 150 can be configured to calculate the crater diameter (D_{c}) using proposed formulas (10) or (11). Since the crater diameter is a function of the gap (Δ), another procedure parameter that may be determined includes the gap between the fiber and the target. This can be determined by the controller 150 and in some instances may be considered to be a typical average gap for fiber lithotripsy (for example, 0.5 mm) and is therefore a value stored in a memory (e.g., memory 140 in FIG. 22) by controller 150. Alternatively, the doctor 120 can input (e.g., via user interface device 135) the gap that he or she requires or intends to use. The crater diameter is also a function of the target threshold (F′). This can be accepted or otherwise determined by controller 150 as an average typical threshold of targeted stones during lithotripsy or a threshold of Bego-stone (for example, 20 J/cm²). Alternatively, a doctor 120 can input a targeted threshold that he or she requires or intends to use during treatment. Another approach can be that the doctor chooses the type of stone (target and target characteristic) at the start of the treatment (e.g. target characteristics such as uric acid, cysteine, calcium oxalate monohydrate (COM), struvite, xanthin, silicate, mixed/combined type of stone etc.) and inputs this information as input parameters (via user interface device 135) and the controller 150 determines the stone threshold using a preset/predetermined value (for example, prior gained/measured threshold data of each stone type, stored in memory 140).

In accordance with another embodiment, a maximum N-factor (Nfmax, also referred to herein as Nmax) is calculated or otherwise determined, and Nfₘₐₓ can be considered to be the most efficient number of pulses, as discussed previously. In process 1800, Nfₘₐₓ is determined (e.g., by controller 150) at step 1810 using formula (14).

In some embodiments, a procedure parameter such as pulse frequency is calculated, which may be based at least in part on Nfmax. For instance, in process 1800 the pulse frequency (f) is calculated at step 1812 using expression (7):

$$f = ( {Nf}_{max} * {D}_{c} ) / υ \qquad ( 15 )$$

In accordance with another embodiment, a procedure parameter such as the pulse energy is calculated. In some embodiments, the pulse energy is calculated (e.g., by controller 150) based at least in part on the pulse frequency. For example, at step 1816 of process 1800 the pulse energy (E) is calculated using the expression:

$$E = \overline{P} / f \qquad ( 16 )$$

The next step at 1816 is to check the safety of the calculated parameters. For example, a procedure parameters, such as the pulse energy E, may be compared (e.g., by controller 150) to a threshold value (e.g., Emax) which corresponds with a safety parameter value.

According to a further embodiment, if the calculated pulse energy E is less than or equal to the threshold Emax, then a second comparison calculation may be performed (e.g., by controller 150) at step 1822. For instance, the calculated pulse energy E can be compared to a second threshold value, such as the average safety pulse energy E calculated at step 1806. If the calculated pulse energy E is greater than the second threshold value (E) by a predetermined amount (e.g., 20%) ((Ē−E)/Ē>0.2), then in some embodiments the process returns to step 1808 to determine (e.g. by controller 150) the crater diameter D_{c} using proposed formulas (10) or (11) using the calculated pulse energy (calculated at step 1814 using expression (16)), and the process continues from step 1808. If the calculated pulse energy E is less than or equal to the second threshold value (Ē) by a predetermined amount (e.g., 20%), then the controller 150 displays one or more of the procedure parameters (whether already calculated or calculated at this step) such as laser operating parameters, on the display device 130 (e.g., pulse energy, power, and frequency). This is shown in process 1800 at step 1824.

Returning to step 1816, if the calculated pulse energy is greater than or otherwise exceeds the threshold (E>Eₘₐₓ), then the controller 150 at step 1818 sets the pulse energy E as the maximum pulse energy (the safety pulse energy value) Emax, and then at step 1820 the pulse frequency is recalculated (e.g., by controller 150) using expression (16). Once the pulse frequency is recalculated, then the controller 150 displays the one or more process parameters (e.g., laser operating parameters, such as pulse energy, pulse frequency, average power, peak power) on the display device 130 (e.g., step 1824 as discussed above).

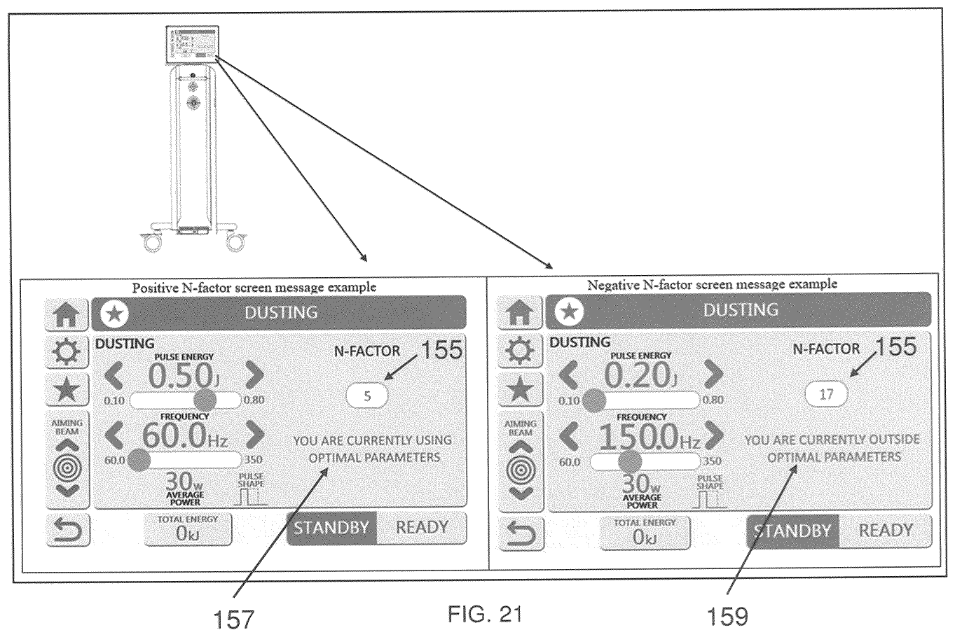

In some embodiments, when the pulse energy E is less than or equal to the threshold value (e.g., Emax), then the controller 150 outputs a positive alert message on the display device 130. An example of such a positive alert message 157 is shown on the left side of FIG. 21 (i.e., “You are currently using optimal parameters”). In alternative embodiments, when the pulse energy E is greater than the threshold value (e.g., Emax), then the controller 150 outputs a negative alert message 159 on the display device 130. An example of such a negative alert message is shown on the right side of FIG. 21 (i.e., You are currently outside optimal parameters”).

In accordance with at least one embodiment, a last step of the process is for a user 120 to authorize the displayed procedure parameters from step 1824. For instance, a doctor at step 1826 may authorize the calculated parameters (e.g., by interacting with the user input device 135 and/or by starting the procedure). The procedure would then commence using these displayed procedure parameters.

### Second Example of a Laser System with Smart Assistant Mode

A flowchart of steps showing an example of a second approach method 1900 using the smart assistant mode is shown in FIG. 19. A first step at 1902 is similar to step 1802 discussed above in reference to process 1800 of FIG. 18), and includes receiving (e.g., by the controller) one or more input parameters as previously described.

In accordance with another embodiment, at least one procedure parameter is determined. For instance, at the second step 1904 in process 1900 comprises determining at least one procedure parameter, such as a determination of the fiber speed (ν) in a similar manner as described above in reference to step 1804 in the first approach method 1800.

The next step is divided into two parallel processes/actions.

The first parallel process/action starts at step 1906 with the doctor inputting (via user input device 135) a pulse energy E that falls within the safety range (e.g., the safety range is based on input safety parameters) that he or she wants to use (E). In some instances this step may be performed by controller 150 based on one or more input parameters. Another procedure parameter that may be calculated includes the crater diameter Dc, which is done at step 1908 using proposed formulas (10) or (11). In some embodiments, the crater diameter Dc at step 1908 may be based at least in part on the input pulse energy E put in at step 1906. This step may also be performed by the controller 150. The fiber gap (4) and target threshold (F′) can be input as constant values at step 1908, in a similar manner as described above. The next step 1910 is to calculate pulse frequency (f) using expression (16), which is performed by controller 150. This is followed by calculating the number of pulses, i.e., N-factor (Nf) using expression (7) at step 1912 (which can also be done by controller 150).

The second parallel process/action starts at step 1914 with a calculation of the average safety energy using a received safety energy range (Ē), in a similar manner as described above in reference to step 1806 of process 1800, which can also be calculated or otherwise determined by controller 150). At step 1916 a procedure parameter such as the crater diameter Dc is calculated (e.g., by controller 150) using proposed formulas (10) or (11) in a similar manner as described above in reference to step 1808 of process 1800. The fiber gap (Δ) and target threshold (F′) can be input as a constant value here, in a manner similar to that described above.

The next step 1918 is to calculate (e.g., by controller 150) the maximum N-factor Nfmax, i.e., the most efficient number of pulses, using formula (14) in a similar manner as described above in reference to step 1810 of process 1800. A procedure parameter such as the pulse frequency (f), is calculated at step 1920 using expression (15) in a similar manner as described above in reference to step 1812 of process 1800. Another procedure parameter such as the pulse energy E using expression (16) is then calculated at step 1922 in a similar manner as described above in reference to step 1814 of process 1800.

At step 1924, a safety check similar to step 1816 of process 1800 is performed for the calculated parameters. For example, a procedure parameter such as the pulse energy E is compared to a threshold value (Emax) by controller 150. If the calculated pulse energy is greater than or otherwise exceeds the threshold value, then the controller 150 at step 1926 sets (adjusts) the pulse energy E to be the maximum pulse energy Emax (E=Emax) and the pulse frequency is recalculated (e.g., using expression (16)) at step 1928 in a similar manner as described above in reference to steps 1818 and 1820 of process 1800. In process 1900, after step 1928, the process returns to step 1918 to calculate the maximum N-factor using formula (14) due to the changed parameters of energy and frequency (E=Emax at step 1926 which triggers a pulse frequency change at step 1928). If and when the calculated pulse energy E does not exceed the upper limit on the safety range (E≤Eₘₐₓ), then the second parallel process/action is over.

The next step 1930 in the process 1900 is to compare N_{f} to Nfmax. For instance, the Nf calculation made at step 1912 is compared to an Nfₘₐₓ value calculated at step 1918. Recall that the crater diameter Dc value calculated at step 1908 that is used to calculate Nf at step 1912 may itself be dependent on a pulse energy value put in by the doctor at step 1906. Although this Nf to Nfmax comparison is explicitly shown in FIG. 19, it is to be appreciated that this step may be included in all the approaches discussed herein.

If Nf is greater than Nfmax, then at step 1934 the controller 150 gives feedback and informs the doctor that he or she is currently outside the optimal laser parameters, e.g., via a positive or negative alert message. This can be implemented using any one of a number of different ways such as (but not limited to) outputting a corresponding warning (notification) on the screen (visual notification) in combination with the current Nf value. FIG. 21 shows two different screens (GUI screen shots) of outputs to the user 120 on a display device 130. The screenshot on the right is an example of a negative alert message 159 (i.e., You are currently outside optimal parameters) indicating to the user 120 that Nf 155 (a value of 17) is greater than Nfₘₐₓ and that optimal procedure parameters (i.e., laser operating parameters) have not been calculated. The sub-optimal procedure parameters may also be displayed on the display device 130 to the user 120. If Nf is less than or equal to Nfmax, then at step 1932 the controller 150 gives positive feedback to the user 120. For instance, the screenshot on the left side of FIG. 21 is one example of a positive alert message (i.e., You are currently using optimal parameters) indicating to the user that the N factor 155 (a value of 5) is less than Nfₘₐₓ and that optimal procedure parameters, including laser operating parameters, have been calculated. The optimal procedure parameters may also be displayed on the display device 130 to the user 120.

According to some embodiments, in response to a determination that Nf is greater than Nfmax, at least one laser operating parameter may be adjusted, as exemplified in step 1924, where the pulse energy is set to be the maximum safety value, which then fed back into the process to calculate a new Nfₘₐₓ value. In some embodiments, in response to a determination that Nf is less than or equal to Nfmax, then the laser source may be controlled using the laser operating parameters.

Although not explicitly shown in process 1900, the controller 150 may display one or more of the procedure parameters (whether already calculated or calculated at this step) such as laser operating parameters, on the display device 130 (e.g., pulse energy, power, and frequency) in a similar manner as described above in reference to step 1824 of process 1800 prior to the doctor authorization step at 1936.

Besides the positive or negative alert messages 157 and 159 shown in FIG. 21, it is to be appreciated that in addition, other visual signals, and/or a voice signal (audio notification) and/or a haptic notification may be provided simultaneously as possible feedback.

The last step 1936 is for a doctor (or a doctor's assistant) 120 to authorize the desired parameters in a similar manner as described above in reference to step 1826 of process 1800. User 120 has to choose here where he or she may still want to use suboptimal laser parameters and to perform a more inefficient procedure, or here is where he or she chooses to accept the proposed calculated (and most efficient) laser parameters. In the alternative, he or she can choose to keep changing laser parameters (for example, pulse energy and/or frequency) that still fall under the condition where Nf≤Nfmax.

### Third Example of Laser System with Smart Assistant Mode

A flowchart showing steps in a third example approach method 2000 using the smart assistant mode is shown in FIG. 20. In accordance with certain aspects, this approach is considered to be a more precise and effective method since it assumes the implementation of a real-time feedback system and one or more laser sensors 125 (e.g. fiber speed, current fiber distance to target, current stone type and/or structure recognition, e.g., sensor 125 in FIG. 22).

A first step at 2002 is similar to step 1802 discussed above in reference to process 1800 of FIG. 18), and includes receiving (e.g., by the controller) one or more input parameters as previously described. Step 2004 comprises a calculation of the average safety energy calculation (Ē) based on a received safety energy range and is similar to step 1806 described previously of process 1800.

The next step is divided into three parallel processes/actions.

The first parallel process/action is acquisition of the current fiber speed data (ν) at step 2006. This can be realized using different methods such as (but not limited to) a relative fiber speed calculation based on a so-called real-time visual image computer processing or similar (e.g., processing an image that includes the treatment zone and the fiber (distal tip)), a built-in fiber speed sensor such as an accelerometer, or other technique. For example, sensor 125 of FIG. 22 may include a sensor configured as a fiber speed sensor. i.e., a sensor configured to measure a fiber speed. If the fiber speed sensor is absent in the lithotripsy system, this current approach still can be used by inputting in this step a fiber speed as a constant value, in a similar manner as described above in reference to steps 1804 of process 1800 and step 1904 of process 1900.

The second parallel process/action at step 2008 includes acquisition of data from a distance sensor (e.g., sensor 125 of FIG. 22 may include a distance sensor)) that measures the current gap between the fiber tip and target surface. This can be realized by different possible ways such as (but not limited to) relative target size calculation based on a relationship between the target size and the fiber diameter (so-called real-time visual image computer processing, or similar, as mentioned above), back reflection/scattering sensor, ultrasound sensor, or other. If the fiber distance sensor is absent in the lithotripsy system and, this current approach can still be used by inputting in this step a constant value for the fiber gap, in a similar manner as described above in processes 1800 and 1900.

The third parallel process/action at step 2010 includes acquisition of data regarding the stone type and/or structure that is currently situated in front of the fiber. This can be accomplished using different techniques, such as (but not limited to), a sensor (e.g., sensor 125 of FIG. 22) configured as a back reflection/scattering sensor, a fluorescent sensor, a visual sensor, or other technique/sensor.

In accordance with at least one embodiment, data (procedure parameters) collected at steps 2006, 2008, and/or 2010 is received by controller 150.

A procedure parameter such as the calculi or stone ablation threshold F′ may then be calculated (e.g., by controller 150) at 2012. This can be accomplished by the controller 150 using a preset value (for example, prior gained/measured threshold data of each stone type) stored in memory 140 and based on the target input parameters. In some embodiments, if the stone type and/or structure sensor is absent in the lithotripsy system, this current approach still can be used by inputting at this step a target threshold as a constant value, in a similar manner as was described above in reference to processes 1800 and 1900.

This step (with the three parallel processes) is over when all the current required sensor's data regarding the procedure parameters and/or target parameters is acquired for all parallel processes/actions of data acquisition.

The next step at 2014 is to check whether the acquired sensor's data differs from that which was previously collected. One or more procedure parameters (e.g., fiber speed, distance between fiber tip and target, target characteristics) are compared to stored procedure parameters by controller 150. Basically the system checks as to whether any changes happened since the last automatic laser parameter settings calculation. In accordance with some aspects, step 2014 may be optional, such as at the beginning of a process. Step 2014 may important in some embodiments because during the treatment the speed of fiber movement and or characteristic as gap between fiber tip and stone can be change and these values need to be re-checked.

If the fiber moves with the same constant speed (ν), the target is the same stone with the same threshold (F′), and it is situated at the same distance (Δ), then there is no need to change any of the previous laser parameters settings (if any). Thus, at step 2014 if the acquired sensor data related to one or more procedure parameters does not differ from that previously collected (or there is not yet previously collected data), then the next step at 2016 is to calculate at least one procedure parameter, such as the crater diameter (D_{c}) using proposed formulas (10) or (11). Otherwise, i.e., if the acquired data (fiber speed, distance between fiber tip and target), and/or target characteristics (ablation threshold) do differ from that previously collected, then the process returns back to the step(s) of acquiring new real-time sensor data (2006, 2008, 2010).

The next step at 2018 is to calculate Nfₘₐₓ using formula (14), which is followed by calculating the pulse frequency (f) at step 2020 using expression (15), and then calculating the pulse energy (E) at step 2030 using expression (16). Each of these steps is similar to steps 1810, 1812, and 1814 respectively as described previously in process 1800 and may be performed by controller 150.

At step 2032 a check of the safety of the calculated parameters is performed in a similar manner as described above in reference to step 1816 of process 1800. As also described above in reference to process 1800 a positive or negative alert message may be displayed on the display device as a result of the comparison between the pulse energy (or Nf) to the threshold value (Emax or Nfmax).

If the calculated pulse energy is less than or equal to the threshold (E≤Eₘₐₓ), then a second comparison calculation is performed at step 2036 in a similar manner as described above in reference to step 1822 of process 1800. For instance, if the calculated pulse E is greater than the second threshold value by a predetermined amount, then in some embodiments the process returns to step 2016 to determine (e.g., by controller 150) the crater diameter in a similar manner as the return to step 1808 of process 1800 described above. If the calculated pulse energy E is less than or equal to the second threshold value, then controller 150 displays one or more of the procedure parameters (whether already calculated or calculated at this step), such as laser operating parameters (e.g., pulse energy, pulse frequency, average power) on the display device 130 at step 2040. These laser operating parameters may be considered to be the proposed or “current” laser operating parameters and according to at least one embodiment, the controller 150 may then control laser source 105 using these laser operating parameters. Process 2000 therefore skips the “doctor authorization” steps 1826 of process 1800 and 1936 of process 1900.

Returning to step 2032, if the calculated pulse energy E is greater than a threshold value (Emax), i.e., E>Emax, then at step 2034 controller 150 sets the pulse energy E as the maximum pulse energy (the safety pulse energy value) Emax, in a similar manner as described above in reference to step 1818 of process 1800, and then at step at 2038 the pulse frequency is recalculated (e.g., by controller 150) using expression (16) (similar to step 1820 of process 1800). Once recalculated, then the controller 150 displays the one or more process parameters (e.g., laser operating parameters such as pulse energy, pulse frequency, average power, etc.) on the display device 130 and then controls laser source 105 using these laser operating parameters (e.g., step 2040 as discussed above).

The last step of this process is to go back to the step(s) of acquiring data from the sensor(s) (steps 2006, 2008, 2010) until the lithotripsy procedure is determined to be over at step 2042. In accordance with certain aspects, a user 120 may determine that a process is over when no more calculi is visible in the scope image sensor, and/or the controller 150 may determine that the procedure is over when no more calculi material is detected in the volume of interest (kidney, ureter, bladder). This whole process is cyclical and consists of real-time sensor's data acquisition and real-time calculation and setting of most efficient current laser parameters based on acquired data.

The faster the sensors 125 are able to work (measure and communicate), the faster the laser parameters can be adapted to real-time changing process parameters (e.g. fiber speed, gap, stone type/structure) and the more efficient the lithotripsy procedure.

### General System Description

FIG. 22 is a block diagram of one non-limiting example of a laser system (also referred to herein as a lithotripsy system), shown generally at 100, that is provided by at least one embodiment that is for use in a lithotripsy procedure. In accordance with various aspects, system 100 can operate in a smart assistant mode of operation in accordance with the flowcharts as described in FIGS. 18, 19, and 20. System 100 comprises a controller 150 coupled to a display device 130 and a user input device 135.

The display device 130 is configured to display information (output) to the user 120 (e.g., a doctor) and the user input device 135 is configured to receive information (input) from the user 120 (e.g., one or more input parameters). In some embodiments, the display device 130 and user input device 135 may be integrated into one device. For instance a graphical user interface (GUI) may be displayed on a touch screen to the user 120 and display information (e.g., the N factor) to the user and receive (via the touch screen) information from the user 120. The controller 150 is coupled to the display device 130 and is configured to display on the display device 130 information such as procedure parameters, laser operating parameters, Nf, and/or Nfmax.

The user input device 135 is configured to receive input from a user 120, such as a doctor, and can take any one of a number of different forms, including a touchscreen. Besides a touch sensitive screen, other non-limiting examples of a user input device include a cursor control device (CCD), such as a mouse, a trackball, or joystick; a keyboard; one or more buttons, switches, or knobs; and a voice input system. In some embodiments, input from the user constitutes user input data that can be used (at least in part) by controller 150 to control one or more components of the system 100, such as the laser source 105.

User input data may include initial user input data that is received from a user 120 at the initiation of a procedure. In some embodiments, the initial user input data includes input parameters, including at least one of (1) one or more properties of the laser lithotripsy system, and (2) one or more properties of the target, and (3) one or more safety parameters.

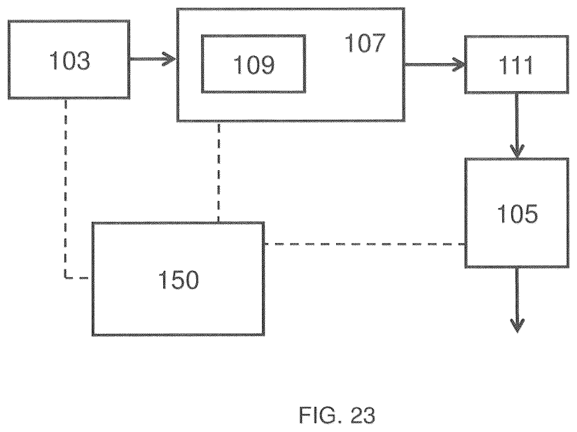

In accordance with at least one embodiment, system 100 also comprises a laser source 105 configured to generate pulsed laser energy. Non-limiting examples for the laser source 105 include a Thulium-doped fiber laser (TFL), an Erbium-doped fiber laser, a Yttrium-doped fiber laser, a Ho:YAG solid-state laser, a Tm:YAG solid-state laser, or an Nd: YAG solid-state laser. Although not explicitly shown in FIG. 22, either controller 150 or the processing laser 110 itself comprises a driver for the laser source. According to one embodiment, the pulsed laser energy is generated using an energy storage device 109, as shown in the configuration shown in FIG. 23. In this configuration, a power supply 103, a laser driver 107, a pump 111, and an energy storage device 109 (e.g., electrical capacitor and/or inductor) are included in system 100. The pump 111 is configured with one or more diode lasers that provide laser radiation to laser source 105. The power supply 103 supplies power to the system and the energy storage device 109 is configured to store a sufficient amount of energy necessary to form a laser pulse. The laser driver 107 of the pump 111 forms an electrical pulse of specified characteristics in response to a control signal from controller 150. The electrical pulses are received by one or more diodes of pump 111 which form an optical pulse necessary to pump the laser medium of laser source 105.

In alternative embodiments, the pulsed laser energy can be generated via any one of a number of techniques, including a laser modulator (e.g., AOM, EOM, EAM), Q-switching, mode-locking, cavity dumping, and/or gain switching.

Returning now to FIG. 22, in accordance with at least one embodiment, system 100 also comprises an optical fiber 110 that is configured to direct the pulsed laser energy at a target 115. The pulse laser energy may be output as laser beam 112 and used to treat any one of a number of urological conditions at the target. According to some embodiments, the optical fiber 110 may be configured as a component of a lithotripsy device. The lithotripsy device (e.g., cystoscope, flexible endoscope with sheaths/no sheaths, (percutaneous nephrolithotomy (PCNL)/rigid endoscope, mini/ultramini PCNL endoscope), may include other associated support components such as fluid flow devices (e.g., irrigation and aspiration functionality), one or more optics, reflective devices, an articulated arm, and/or mechanical devices or robotic devices that are configured to assist with performing the lithotripsy procedure.

The controller 150 is coupled to the laser source 105, the user input device 135, and the display device 130, as well as (optionally) one or more sensors 125 (as discussed above in reference to procedures 1800, 1900, and 2000). Although only one sensor 125 is shown in FIG. 22, it is to be appreciated that more than one sensor may be used in the lithotripsy system. In accordance with at least one embodiment, the sensor 125 is configured to measure at least one input parameter and/or at least one procedure parameter. It is also appreciated that sensor 125 may be a video sensor of the treatment area, for example as part of the endoscope, and the processed endoscopic video can be used for calculation of the fiber tip velocity and/or fiber gap distance, as well as for calculation of the N-factor in real time, and/or adjustments to laser parameters (as described in reference to FIG. 20, which is the automated algorithm). It is to be appreciated that the endoscope may include a processor that is configured to determine these values, but in the interest of simplicity, controller 150 of FIG. 22 is used here to describe this functionality. Fiber speed can be measured using other various methods and devices, such as a speed sensor integrated in the distal tip of the endoscope.

The controller 150 includes circuitry that may be separate or integral components. It will be appreciated by those skilled in the art that the operations performed by the controller 150 may be performed by one or more controllers, processors, and/or other electronic components, including software and/or hardware components. For example, controller 150 includes a processor 145 (which may include more than one processor, also referred to as a central processing unit CPU as understood by those skilled in the art) and a computer-readable-storage device (not explicitly shown in FIG. 22), and a memory 140 (also referred to as a storage device or storage memory), as well as other hardware and software components as will be appreciated by those of skill in the art.

Processor 145 may be a single core or multi core processor, or a plurality of processors for parallel processing and can execute a sequence of machine-readable instructions, which can be embodied in a program or software. The instructions may be stored in a memory location, such as memory 140. The instructions can be directed to the processor 145, which can subsequently program or otherwise configure the processor 145 to implement methods and/or steps of the present disclosure.

Storage memory 140 includes one or more computer-readable and/or writable media, and may include, for example, a magnetic disc (e.g., a hard disk drive HDD), an optical disc (e.g., a DVD, a Blu-ray®, or the like), a magneto-optical disk, semiconductor memory (e.g., a non-volatile memory card, Flash® memory, a solid state drive, SRAM, DRAM), an EPROM, an EEPROM, etc. Storage memory 140 may store computer-readable data and/or computer-executable instructions including Operating System (OS) programs, and control and processing programs.

Electronically stored information is stored in memory 140 of controller 150. In accordance with at least one embodiment, the electronically stored information may comprise look-up tables, empirical functions, and/or analytical models. In some embodiment, this information is generated by inputting results from pre-clinical and clinical tests, trials and studies.

The controller 150 can display data (e.g., one or more procedure parameters, Nf, Nfmax, etc.) on the display device 130. The display device may provide three dimensional or two dimensional images and non-limiting examples include touch screen displays and/or flat panel displays or any other suitable visual output device capable of displaying graphical data and/or text to the user. In some embodiments a touch screen may function as both the display device 130 and as the user input device 135. According to at least one embodiment, the controller 150 is configured to generate on the display device 130 a graphical user interface (GUI) that receives user input in conjunction with the user input device 135.

The system and user interfaces disclosed herein may enable physicians and technicians to make smarter and quicker decisions to enhance the outcome of lithotripsy procedures (e.g., optimize stone removal), reduce procedure time, and reduce cognitive loading required during procedures. This is true especially when the system is more fully automated with control/monitoring using a computer as described above in the algorithm of FIG. 20 which provides the most accurate control of laser parameters and N-factor and shortest procedure time.

### Robotic System

In some embodiments joy stick control of a mechanical arm that controls fiber position and movement is considered. The clinician controls insertion and positioning of the device via joy stick controls that more precisely move and position the fiber and, possibly, the endoscope together. Speed and position of fiber can now directly be measured via sensors monitoring the electronic/pneumatic signals to/from the mechanical arm/joy stick control system. This information can be utilized in the laser control portion of the algorithm discussed above in this disclosure. In addition images from the endoscopic camera can be processed by an image processing algorithm that can, for example, assesses stone type, validate fiber speed and position (used in conjunction with the electronic sensing signals mentioned above), and detect unsafe conditions in the treatment zone such as a fiber directed to an unsafe target. The laser control module will receive data to adjust the laser parameters discussed above in this disclosure. As mentioned previously, and referencing FIG. 22, the display device 130 and user input device 135 may be combined into a single subsystem, e.g., a computer applicable to this monitoring and control system. The computer algorithm can perform calculations in real time to update laser parameters, assess unsafe conditions and provide corrective feedback in the case of detected, erroneous fiber movements in times much faster than is humanly possible.

The aspects disclosed herein in accordance with the present invention, are not limited in their application to the details of construction and the arrangement of components set forth in the following description or illustrated in the accompanying drawings. These aspects are capable of assuming other embodiments and of being practiced or of being carried out in various ways. Examples of specific implementations are provided herein for illustrative purposes only and are not intended to be limiting. In particular, acts, components, elements, and features discussed in connection with any one or more embodiments are not intended to be excluded from a similar role in any other embodiments.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. In addition, in the event of inconsistent usages of terms between this document and documents incorporated herein by reference, the term usage in the incorporated reference is supplementary to that of this document; for irreconcilable inconsistencies, the term usage in this document controls. Moreover, titles or subtitles may be used in the specification for the convenience of a reader, which shall have no influence on the scope of the present invention.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

1. A system for use in a laser lithotripsy procedure, comprising:
a laser source configured to generate pulsed laser energy;
an optical fiber configured to direct the pulsed laser energy at a target; and
a controller coupled to the laser source and configured to:
receive one or more input parameters;
determine at least one procedure parameter, wherein the at least one procedure parameter includes one or more laser operating parameters;
calculate an N-factor (N_{f}) based at least in part on one or more procedure parameters and at least one input parameter, where N_{f} corresponds to a number of laser pulses delivered to a treatment zone having a diameter approximately equal to a diameter of a laser crater (D_{c}) created by the pulsed laser energy that is directed at the treatment zone on the target;
compare Nf to a maximum Nf (Nfmax); and
in response to a determination that Nf is greater than Nfmax, adjust at least one laser operating parameter of the one or more laser operating parameters, or
in response to a determination that Nf is less than or equal to Nfmax, control the laser source using the one or more laser operating parameters.

2. The system of claim 1, wherein the one or more laser operating parameters comprise pulse energy, pulse frequency, average power, peak power, and/or pulse width.

3. The system of claim 1, wherein when the controller determines that Nf is greater than Nfmax, the controller is configured to adjust the at least one laser operating parameter such that Nf is less than or equal to Nfmax.

4. The system of claim 1, wherein the controller is further configured to:
display Nf on a display device coupled to the controller; and
in response to the determination that Nf is greater than Nfmax, output a negative alert message on the display device, or
in response to the determination that Nf is less than or equal to Nfmax, output a positive alert message on the display device.

5. The system of claim 1, wherein the one or more procedure parameters used to calculate Nf include laser pulse frequency, laser crater diameter (Dc), and an optical fiber speed

9. The system of claim 8, wherein the K value is in a range of about 25-75% inclusive.

10. (canceled)

11. The system of claim 1, wherein the controller is configured to calculate Nfmax, wherein Nfmax is calculated according to the formula:
$$Nfmax = ( 12.4 - 3.4 d ) + ( 3.3 d - 8.4 ) / ( 1 + 4.7 \times 1 {0}^{- 5} exp ( {P}_{peak} / 0.0375 ) ) - ( ( 14.7 d - 16 ) + ( 80 - 65 d ) {P}_{peak} + ( 55 d - 62 ) {P}_{peak}^{2} ) / ( 1 + 0.34 {E}^{5.5 - 2.6 Ppeak} ) ,$$
where Pₚₑₐₖ is peak power.

12. The system of claim 1, wherein Nfₘₐₓ is in a range of 1-12 inclusive.

13. (canceled)

14. The system of claim 12, wherein Nfₘₐₓ is in a range of 1-5 inclusive.

15. (canceled)

16. The system of claim 1, wherein the controller is configured to display on a display device at least one laser operating parameter based on the comparison.

17. The system of claim 1, wherein the controller is configured to display on a display device at least one of Nf and Nfmax.

18. The system of claim 1, further comprising at least one sensor coupled to the controller and configured to measure at least one input parameter and/or at least one procedure parameter.

19. The system of claim 18, wherein the at least one sensor includes a sensor configured to measure a fiber speed.

20. The system of claim 19, wherein when the controller determines that Nf is greater than Nfmax, the controller is configured to adjust the at least one laser operating parameter such that Nf is less than or equal to Nfmax.

21. The system of claim 1, wherein the controller is further configured to:
in response to a determination that Nf is greater than Nfmax, output a negative alert message that comprises at least one of an audio alert message, a visual alert message, and a haptic alert message, or
in response to a determination that Nf is less than or equal to Nfmax, output a positive alert message that comprises at least one of an audio alert message, a visual alert message, and a haptic alert message.

22. The system of claim 1, wherein the controller is configured to output an alert message that comprises at least one of an audio alert message, a visual alert message, and a haptic alert message in response to the comparison.

23. The system of claim 22, further comprising a user input device that is coupled to the controller, wherein the user input device is configured to receive input from a user, the input including at least one value for a laser operating parameter that is used by the controller to control the laser source.

24. The system of claim 23, wherein the alert message includes information to the user regarding whether Nf is greater than Nfmax or whether Nf is less than or equal to Nfmax.

25. The system of claim 1, wherein the input parameters comprise at least one of target parameters, system parameters, and safety parameters.

26. The system of claim 25, wherein the input parameters include target parameters and the target parameters comprise target type, target location, and one or more target characteristics.

27. The system of claim 26, wherein the target is calculi and the one or more target characteristics comprise size and/or hardness of the calculi.

28. The system of claim 27, wherein the input parameters include at least one system parameter and the at least one system parameter comprises a fiber diameter and/or a fiber numerical aperture.

29. The system of claim 23, wherein the input parameters include at least one safety parameter and the at least one safety parameter comprises maximum average power, maximum peak power, maximum pulse energy, and/or maximum pulse frequency.

30-51. (canceled)
